(19) United States
(12) Patent Application Publication
(10) Pub. No.: US 2010/0227323 A1
(43) Pub. Date: Sep. 9, 2010

(54) MICROCHANNEL DETECTION DEVICE AND USE THEREOF

(75) Inventors: Antje Baeumner, Ithaca, NY (US); Sam R. Nugen, Ithaca, NY (US)

Correspondence Address: NIXON PEABODY LLP - PATENT GROUP, 1100 CLINTON SQUARE, ROCHESTER, NY, 14604, US

(73) Assignee: CORNELL RESEARCH FOUNDATION, INC., Ithaca, NY (US)

(21) Appl. No.: 12/670,893

(22) PCT Filed: Jul. 30, 2008
(86) PCT No.: PCT/US08/71603

§ 371 Date: May 19, 2010

Related U.S. Application Data
(60) Provisional application No. 60/952,724, filed on Jul. 30, 2007.

(51) Int. Cl. C12Q 1/68; G01N 30/96; G01N 33/543; G01N 33/53; C12M 1/34; B05D 3/14

(52) U.S. Cl. 435/6; 422/69; 436/518; 435/7.2; 435/287.1; 435/287.2; 427/540

(57) ABSTRACT

The present invention relates to a device and methods for detecting or quantifying an analyte in a test sample. The device includes a substrate defining one or more microchannels and having a reaction region in a first portion of the one or more microchannels, wherein the reaction region contains a first binding element selected to bind with a first portion of the analyte. The device also includes a detection region in fluid communication with the reaction region. The detection region includes a second binding element selected to immobilize the analyte within the detection region. Methods of detecting or quantifying an analyte in a test sample using the device of the present invention are also disclosed. A method for coating a polymer with a gold layer is also disclosed.

This application claims the benefit of U.S. Provisional Patent Application Ser. No. 60/952,724, filed Jul. 30, 2007, which is hereby incorporated by reference in its entirety.

This invention was made with government support under NYSTAR grant number C040052 awarded by the National Science Foundation. The government has certain rights in this invention.

### FIELD OF THE INVENTION

The present invention relates to a microchannel device for the detection or quantification of analyte in a sample and methods of use thereof. Methods of using the device may employ marker-loaded particles, e.g., liposomes, and either electrochemical or optical detection of a target analyte in a test sample. The present invention also relates to a method for coating a polymer with gold.

### BACKGROUND OF THE INVENTION

Cryptosporidium parvum is a parasitic protozoan that continues to be a significant problem in the water industry. In addition to being found in municipal water supplies, C. parvum can also survive common chlorination treatments used by public pools and water parks. The current EPA methods for C. parvum detection rely on trained technicians to visually identify stained oocysts. This process is both laborious and time intensive.

Nucleic acid detection methods are potentially useful for detecting and measuring the presence of organisms, such as pathogens, in food and water supplies. Southern, northern, dot blotting, reverse dot blotting, and electrophoresis are the traditional methods for isolating and visualizing specific sequences of nucleic acids. Each has advantages and disadvantages. For example, gel electrophoresis, often performed using ethidium bromide staining, is a relatively simple method for gaining fragment length information for DNA duplexes. This technique provides no information on nucleotide sequence of the fragments, however, and ethidium bromide is considered very toxic, although safer stains have been developed more recently.

If, in addition to length information, there is a desire to determine the presence of specific nucleotide sequences, either Southern blotting, for DNA, or northern blotting, for RNA, may be chosen. These procedures first separate the nucleic acids on a gel and subsequently transfer them to a membrane filter where they are affixed either by baking or UV irradiation (a method that often takes several hours). The membrane is typically treated with a pre-hybridization solution, to reduce non-specific binding, before transfer to a solution of reporter probe. Hybridization then takes place between the probe and any sequences to which it is complementary. The initial hybridization is typically carried out under conditions of relatively low stringency, or selectivity, followed by washes of increasing stringency to eliminate non-specifically bound probe and improve the signal-to-noise ratio.

Originally, probes were often labeled with ³²P which was detected by exposure of the membrane to photographic film. Today, however, many researchers are making use of non-isotopic reporter probes. These blotting procedures require more time and effort than simple gel electrophoresis, particularly when low levels of nucleic acid are present. In particular, the entire process to detect a specific sequence in a mixture of nucleic acids often takes up to two days, and is very labor intensive and expensive.

There are a wide variety of DNA and RNA detection schemes in the literature, many of which are available as commercial kits. Nucleic acid detection schemes have seen the same trends in assay design as immunoassays, with efforts directed towards simpler, more rapid, and automatable detection schemes.

Liposomes are of interest as detectable labels in hybridization assays because of their potential for immediate signal amplification. Liposomes are spherical vesicles in which an aqueous volume is enclosed by a bilayer membrane composed of lipids and phospholipids (New, Liposomes: A Practical Approach, IRL Press, Oxford (1990)). Previous studies (Plant et al., Anal. Biochem., 176:420-426 (1989); Durst et al., In: GBF Monograph Series, Schmid, Ed., VCH, Weinheim, FRG, vol. 14, pp. 181-190 (1990)) have demonstrated the advantages of liposome-encapsulated dye over enzymatically produced color in the enhancement of signals in competitive immunoassays. The capillary migration or lateral flow assays utilized in these experiments, avoid separation and washing steps and long incubation times and attain sensitivity and specificity comparable to enzyme-linked detection assays. Nevertheless, for each pathogenic organism, new liposomes and membranes have to be developed. This is a laborious and time-consuming process.

Accordingly, there remains a need for a simple, reliable biosensor that can reduce the time, labor, and cost of detecting environmental and food contaminants, including pathogenic organisms. The present invention is directed to overcoming these and other deficiencies in the art.

### SUMMARY OF THE INVENTION

The present invention relates to a microchannel device for the detection or quantification of an analyte in a sample. This device includes a substrate defining one or more microchannels and having a reaction region in a first portion of the one or more microchannels, wherein the reaction region includes a first binding element selected to bind with a first portion of the analyte. The device also includes a detection region in fluid communication with the reaction region. The detection region includes a second binding element selected to immobilize the analyte within the detection region.

The present invention also relates to a method of detecting or quantifying an analyte in a test sample. This method involves providing a device including a substrate defining one or more microchannels and having a reaction region in a first portion of the one or more microchannels; and a detection region in fluid communication with the reaction region. A test sample potentially comprising a target analyte is introduced into the reaction region, under conditions effective to permit binding between a first binding element present within the reaction region and a first portion of the analyte. The method further includes providing a second binding element selected to immobilize the analyte within the detection region, wherein the second binding element is capable of binding with a second portion of the analyte or a portion of the first binding element and contacting the test sample with the detection region, under conditions effective to immobilize the analyte within the detection region. One or more reporter complexes are provided under conditions effective to permit binding between the reporter complexes and a third portion of the analyte or a portion of the first binding element or a portion of the second binding element. The method further includes detecting any reporter complexes bound to the analyte or first binding element or second binding element in the detection region, and correlating the presence or quantity of bound reporter complexes to the presence or quantity of analyte in the test sample.

A further embodiment of the present invention relates to a method of detecting or quantifying an analyte in a test sample. This method involves providing a device including a substrate defining one or more microchannels and having a reaction region in a first portion of the one or more microchannels and a detection region in fluid communication with the reaction region. A test sample potentially comprising a target analyte is introduced into the reaction region. The method further includes providing one or more reporter complexes, wherein the reporter complexes comprise a first binding element and a marker, under conditions effective to permit binding between the first binding element and a first portion of the analyte and providing a second binding element selected to immobilize the analyte within the detection region, under conditions effective to permit binding between the second binding element and a second portion of the analyte or a portion of the one or more reporter complexes. In addition, the method includes contacting the test sample and the one or more reporter complexes with the detection region, under conditions effective to immobilize the analyte within the detection region, detecting any reporter complexes bound to the analyte in the detection region, and correlating the presence or quantity of bound reporter complexes to the presence or quantity of analyte in the test sample.

Yet another embodiment of the present invention relates to a method for coating a polymer with a gold layer. This method involves providing a polymer having at least a portion of a surface having a plurality of carboxylic acids, conjugating a heterobifunctional molecule including an amino group and a thiol group to the surface under conditions effective to produce a thiolated surface, and adhering a gold layer to the thiolated surface.

The use of a detection device in accordance with the present invention with the capability of specific genetic amplification and detection has the potential for reducing the time, labor, and cost of pathogen detection. Disposable microfluidic devices can be rapidly made at low cost using hot embossing techniques. The use of such rapid biosensors for the detection of pathogens will play a major role in the future of food and water safety.

### BRIEF DESCRIPTION OF THE DRAWINGS

FIG. 1 shows a design of a detection device of the present invention. Insert (i) shows microfluidic mixers arranged in microfluidic channels present in the reaction region of the device. The structures were hot embossed into polymethyl methacrylate (PMMA) using a copper master. The channels were approximately 1 m in length with a volume of about 3.5 μl and a surface area of about 3.5 cm². Insert (ii) shows the detection region of the device having fluorescently tagged and immobilized DNA capture probes.

FIGS. 2A-D show microfluidic mixing in accordance with the present invention. The employed static mixer structure and its mixing characteristics are shown. FIG. 2A shows a sawtooth mixer design which is repeated throughout the microchannel. FIG. 2B shows an analysis of mixing effect across the microchannel width. Pixel intensities are measured and plotted as a function of location within the cross section of the microchannel. FIG. 2C shows a FLUENT software analysis of fluid streamlines. FIG. 2D shows the effect of mixing two solutions in a sawtooth mixer-containing channel and in a straight channel.

FIG. 3A shows oligo (dT)₂₅ magnetic beads dispersed in sawtoothed channels during analyte isolation. FIG. 3B shows the oligo (dT)₂₅ magnetic beads upon application of a magnet.

FIGS. 4A-D show microfabrication of a detection device of the present invention. FIG. 4A is a schematic showing the electroplating of a metal master. FIG. 4B is a schematic showing hot embossing of a polymer substrate. FIG. 4C is a scanning electron micrograph (SEM) of an electroplated copper master. FIG. 4D is an SEM of a hot embossed PMMA substrate.

FIG. 5 is a schematic of nucleic acid sequence based amplification (NASBA).

FIG. 6 shows the detection of amplified RNA in accordance with the present invention using dye encapsulating liposomes.

FIG. 7 shows surface modification of PMMA. The surface of PMMA was initially carboxylated using 12 mW/cm² of UV at 254 nm for seven minutes. The carboxylic acids were then conjugated to cystamine using EDC/sulfo-NHS. The thiolated surface could then provide adequate adhesion for a gold electrode.

FIG. 8 shows electrode formation on PMMA. In Step (a), the PMMA surface is cleaned and thiolated. In Step (b), gold is evaporated on the surface to a thickness of 200 nm. In Step (c), S1827 positive photoresist is spun onto the gold and baked. In Step (d), the resist is exposed and developed leaving the electrode pattern. In Step (e), the PMMA is placed in gold etch to remove the gold not protected by the photoresist. Then, in Step (f), the PMMA is UV treated and the remaining resist is removed with developer.

FIG. 9A is an SEM of a gold interdigitated ultramicroelectrode array (IDUA) formed on a PMMA substrate. FIG. 9B shows a PMMA sheet containing a hot embossed channel which was then bonded to the PMMA containing the IDUA. The finished device contained a 500 μm channel positioned along the IDUA. FIG. 9C shows the finished device containing two channels.

FIG. 10 is a graph showing the dose response of an IDUA to increasing solutions of potassium ferro/ferricyanide flowing through the channel. The potassium ferro/ferricyanide was dissolved in a pH 7.5 phosphate buffer and pumped over the channel at 5 μL/min. Error bars represent the standard deviation of three replicates.

FIG. 11A is a graph showing electrochemical response following the injection of n-Octyl-β-D-glucopyranoside (OG) and lysing of immobilized liposomes. The area of the shaded region was determined and used as the assay result. FIG. 11B is a graph showing the response from assays using NASBA amplicon from 0, 1, 3, and 5 C. parvum oocysts. Error bars represent the standard deviation of a minimum of three replicates.

FIG. 12 shows a microchannel detection device of the present invention having one microchannel. The final device had dimensions of 1.0 cm×4.5 cm. The inlet port can be seen on the right. The two outlets can be seen on the left end.

FIGS. 13A-B show detection of DNA in a PMMA microchannel device. FIG. 13A shows immobilized dendrimers tagged with a capture probe for a sandwich assay with the target analyte and dye containing liposomes. In FIG. 13B, detergent is passed through the channel resulting in lysis of the liposomes and release of the fluorescent dye.

FIGS. 14A-C show the surface properties of a functionalized silicon surface. The silanol surface was established by a hot piranha treatment. The APTMS was conjugated to the silanol surface in an ethanol solution. The carboxyl terminated dendrimer was then conjugated to the APTMS using water soluble carbodiimide chemistry. Error bars represent the standard deviation of a minimum of three measurements.

FIG. 15 is a graph showing surface oxidation of PMMA. The values represent the surface carboxylic acid formation on PMMA as quantified using a toluidine blue O (TBO) dye assay. All treatments had a ten minute duration. Error bars represent the standard deviation of a minimum of four replicates.

FIG. 16 is a graph showing surface carboxylic acid formation on PMMA as a function of UV treatment time. The PMMA was 10 mm from the UV source (10 mW/cm² at 254 nm and 185 nm). The carboxylic acids were quantified with a TBO dye assay. Error bars represent the standard deviation of four replicates.

FIG. 17 is a graph showing the effect of UV treatment on the water contact angle of PMMA. An eight minute duration of UV was sufficient to lower the water contact angle of native PMMA from approximately 65° to 23°. Following a ten minute rinse in DI water, the water contact angle climbed to approximately 48° suggesting the removal of low molecular weight, water soluble polymers created during the UV treatment. Error bars represent the standard deviation of a minimum of four replicates.

FIG. 18 is a graph showing water contact angles of PMMA with varying treatments of oxygen plasma. Oxygen plasma treatment was 150 sccm at 200 W. Error bars represent standard deviations of three replicates.

### DETAILED DESCRIPTION OF THE INVENTION

The present invention relates to a microchannel device for the detection or quantification of an analyte in a sample. This device includes a substrate defining one or more microchannels and having a reaction region in a first portion of the one or more microchannels, wherein the reaction region includes a first binding element selected to bind with a first portion of the analyte. The device also includes a detection region in fluid communication with the reaction region. The detection region includes a second binding element selected to immobilize the analyte within the detection region.

By “analyte” is meant the compound or composition to be measured or detected. It is capable of binding to one or both of the first and second binding elements. Suitable analytes include, but are not limited to, antigens (e.g., protein antigens), haptens, cells, and target nucleic acid molecules. A preferred analyte is a target nucleic acid molecule. A more preferred analyte is a target nucleic acid molecule found in an organism selected from the group consisting of bacteria, fungi, viruses, protozoa, parasites, animals (e.g., humans), and plants. Suitable organisms include, but are not limited to, Cryptosporidium parvum, Escherichia coli, Bacillus anthracis, Dengue virus, and Human immunodeficiency virus (HIV-1).

The binding element includes a “binding material,” by which is meant a bioreceptor molecule such as an immunoglobulin or derivative or fragment thereof having an area on the surface or in a cavity which specifically binds to and is thereby defined as complementary with a particular spatial and polar organization of another molecule—in this case, the analyte. Suitable binding materials include antibodies, antigens, nucleic acid molecules, aptamers, cell receptors, biotin, streptavidin, and other suitable ligands. When the analyte is a target nucleic acid molecule, the first binding element can be a nucleic acid molecule (e.g., capture probe, selected to hybridize with a portion of the target nucleic acid molecule) and the second binding element can be a nucleic acid molecule (e.g., detection probe, selected to hybridize with a separate portion of the target nucleic acid molecule). In a preferred embodiment, the analyte is a target nucleic acid and the first binding element is a target-specific oligonucleotide capture probe and the second binding element is a target-specific oligonucleotide detection probe. The capture and detection probes include a nucleic acid molecule that is specific for separate portions of the target nucleic acid molecule. Such capture and detection probes include, e.g., DNA or peptide nucleic acid (PNA) sequences specific for the target nucleic acid molecule.

Antibody binding materials can be monoclonal, polyclonal, or genetically engineered (e.g., single-chain antibodies, catalytic antibodies) and can be prepared by techniques that are well known in the art, such as immunization of a host and collection of sera, hybrid cell line technology, or by genetic engineering. The binding material may also be any naturally occurring or synthetic compound that specifically binds the analyte of interest.

The first and second binding elements may be selected to bind specifically to separate portions of the analyte. For example, when the analyte is a nucleic acid sequence, it is necessary to choose probes for separate portions of the target nucleic acid sequence. Techniques for designing such probes are well-known. Probes suitable for the practice of the present invention must be complementary to the target analyte sequence, i.e., capable of hybridizing to the target, and should be highly specific for the target analyte. The probes are preferably between 17 and 25 nucleotides long, to provide the requisite specificity while avoiding unduly long hybridization times and minimizing the potential for formation of secondary structures under the assay conditions. Thus, in this embodiment, the first binding element is a capture probe, which is selected to, and does, hybridize with a portion of target nucleic acid sequence. The second binding element, referred to herein as a detection probe for the nucleic acid detection/measurement embodiment, is selected to, and does, hybridize with a portion of target nucleic acid sequence other than that portion of the target with which capture probe hybridizes. The detection probe may be immobilized in the detection region of the device, as described below. Techniques for identifying probes and reaction conditions suitable for the practice of the invention are described in Sambrook et al., Molecular Cloning: A Laboratory Manual, Cold Spring Harbor Laboratory Press (1989), which is hereby incorporated by reference in its entirety. A software program known as “Lasergene”, available from DNASTAR, may optionally be used.

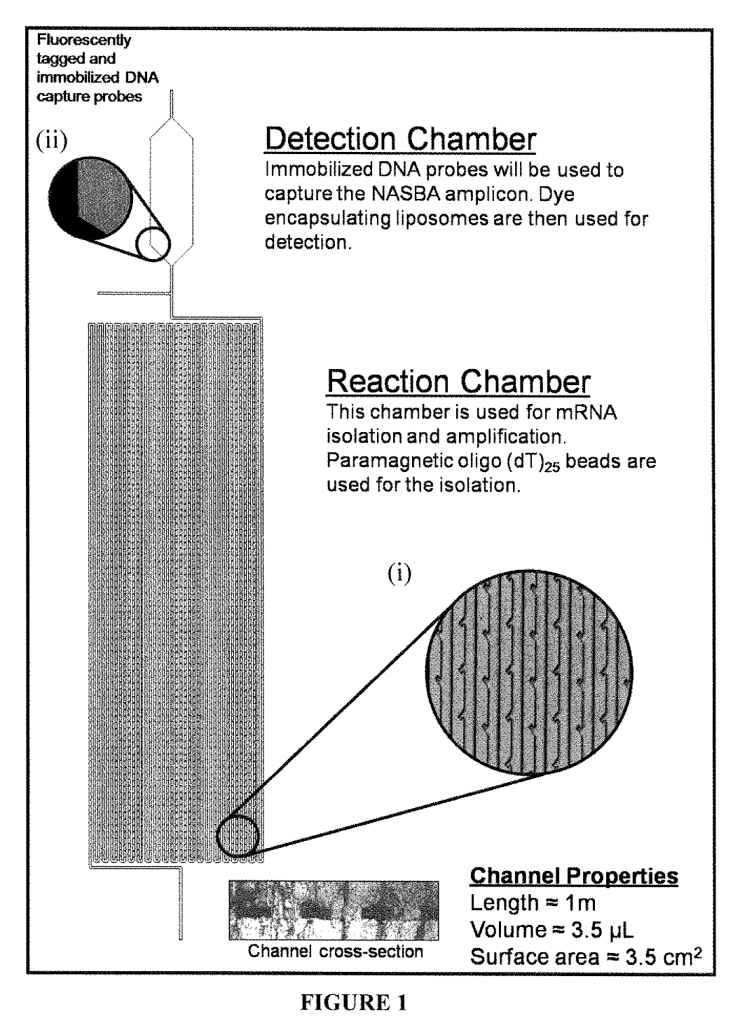

As shown in FIG. 1, a device in accordance with the present invention includes a reaction region. The reaction region is used to allow binding between the first binding element and the analyte and removal of non-bound material. In a preferred embodiment, the analyte is a target nucleic acid and the reaction region is used for nucleic acid isolation and amplification.

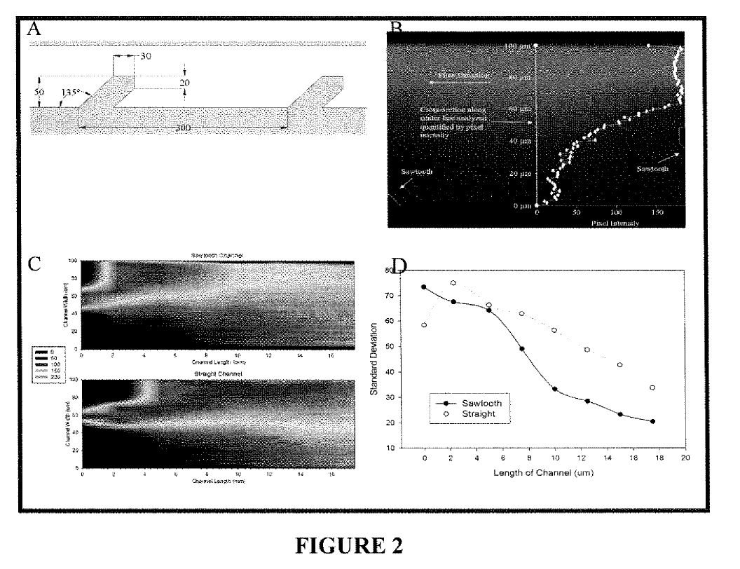

In the present invention, the reaction region is present in a first portion of the substrate defining one or more microchannels. As shown in insert (i) of FIG. 1, in this embodiment, the microchannel includes a sawtooth micro fluidic mixer. The micro fluidic mixer increases solution exposure to the first binding element (and other components) within the reaction region. In particular, as shown in FIGS. 2A-D, a solution pumped through channels containing sawtooth structures achieves a more uniform fluorescence after a shorter length of travel when compared to the same solution flowing through a channel without sawtooth structures. These results suggest the migration of fluorescent molecules is faster in channels containing sawtooth structures.

In one embodiment, the microchannel height and width are less than 150 μm each, more preferably from about 25 μm to about 150 μm. The microchannel(s) can be arranged in any desired pattern. In one embodiment, the microchannel(s) are arranged in a serpentine pattern in order to minimize the overall device dimensions. In another embodiment, the microchannel(s) are arranged in a concentric spiral pattern.

The first binding element is present within the one or more microchannels in the reaction region. The first binding element can optionally be immobilized within the reaction region.

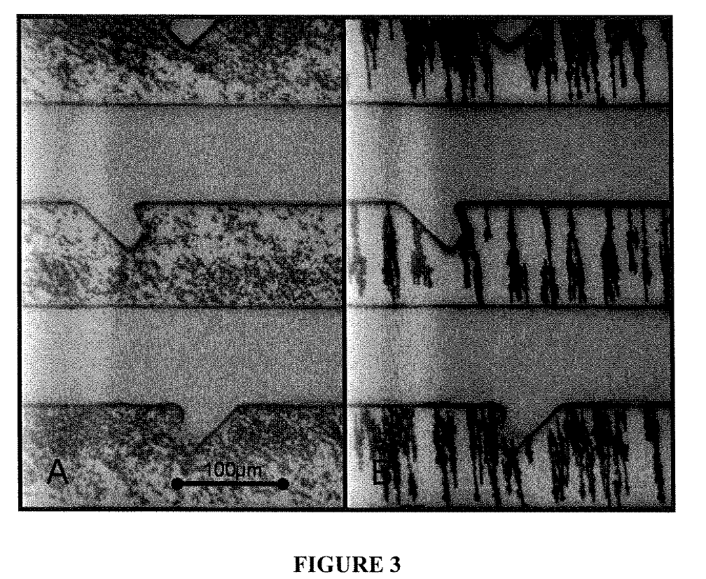

In one embodiment, the first binding element is immobilized to a surface of the reaction region (i.e., a surface within a microchannel). In embodiments in which the first binding element is not immobilized to a surface of the reaction region, the first binding element may include a retention portion that facilitates retention of the first binding element within the reaction region during use of the device. For example, the retention portion can include, but is not limited to, a magnetic bead (e.g., a superparamagnetic bead), polystyrene bead, latex bead, biotin, streptavidin, antibody, a generic nucleic acid sequence, a dendrimer, or a polymer. In a preferred embodiment, the first binding element includes a magnetic bead. During use, the first binding element can be retained within the reaction region of the device by exposing the reaction region to a magnetic field, e.g., by contacting the substrate near the reaction region with a magnet. An example of the use of capture probes attached to magnetic beads is shown in FIGS. 3A-B. As shown in FIG. 3B, when a magnet is positioned proximate the channels, the beads align and are held in place while a sample potentially containing target analyte is introduced or the reaction region is washed of non-bound components.

The second binding element of the present invention is configured to bind to a second portion of a target analyte or a portion of the first binding element (which is bound to the analyte) or to a portion of a reporter complex (see description below). In one embodiment, the test device and methods of the present invention include immobilizing the second binding element in the detection region. As described above with regard to the first binding element, the second binding element may be immobilized to a surface of the detection region or may include a retention portion to facilitate retention of the second binding element within the detection region during use of the device. In one embodiment, the second binding element includes a magnetic bead. In addition, an electromagnetic can be used or a magnet can be moved in and out of the reaction and detection zones to move the first or second binding elements which include magnetic beads within the device. In another embodiment, the second binding element is immobilized onto electrodes in an electrochemical detection device, as described below. The second binding element is capable of binding to a second, separate portion of the analyte or a portion of the first binding element or to a portion of a reporter complex as test mixture passes through the detection region of the device.

Techniques for immobilizing the first and second binding elements to the substrate and/or retention portion will be apparent to the skilled artisan and are dependent on the binding element and substrate/retention portion being used. Suitable coupling groups may be used for immobilization. By “coupling group” is meant any group of two or more members each of which are capable of recognizing a particular spatial and polar organization of a molecule, e.g., an epitope or determinant site. Suitable coupling groups in accordance with the invention include, but are not limited to, antibody-antigen, receptor-ligand, biotin-streptavidin, sugar-lectins, and complementary oligonucleotides, such as complementary oligonucleotides made of RNA, DNA, or PNA (peptide nucleic acid). For example, an antibody, sufficiently different in structure from the analyte of interest, can be employed as a member of a coupling group for a suitably derivatized binding element (i.e., derivatized with the specific antigen of the antibody). Illustrative members of the coupling groups include avidin, streptavidin, biotin, anti-biotin, anti-fluorescein, fluorescein, antidigoxin, digoxin, anti-dinitrophenyl (DNP), DNP, generic oligonucleotides (e.g., substantially dC and dG oligonucleotides), and the like. A preferred method for binding nucleic acid capture probes/detection probes to the substrate is a DMSO-mediated Sn2 reaction with aminated DNA. In addition, spacers can be used to immobilize the first and second binding elements. Suitable spacers include, but are not limited to, poly(ethylene glycol), self-assembled monolayers, and the like.

In a preferred embodiment, the second binding element is a detection probe and the detection probe is attached to the substrate via dendrimers, thereby increasing the amount of surface area available for binding between detection probes and analyte. Dendrimers are homo-poly-functional linkers used as a tether between the surface of the substrate and immobilized DNA. Suitable dendrimers for use in the present invention are described, for example, in Tomalia et al., “Starburst Dendrimers: Molecular-Level Control of Size, Shape, Surface Chemistry, Topology, and Flexibility from Atoms to Macroscopic Matter,” Angew. Chem. Int. Ed. Engl. 29:138-175 (1990); Tomalia et al., “Dendritic Polymers, Divergent Synthesis (Starburst Polyamidoamine Dendrimers),” Polymeric Materials Encyclopedia, Vol. 3(D-E), J. C. Salamone, Ed., CRC Press, New York pp. 1814-1830 (1996), which are hereby incorporated by reference in their entirety. Methods of using dendrimers in microarrays are described, for example, in Le Berre et al., “Dendrimeric Coating of Glass Slides for Sensitive DNA Microarrays Analysis,” Nucleic Acids Res., 31(16):e88 (2003); Mark et al., “Dendrimer-functionalized Self-assembled Mono layers as a Surface Plasmon Resonance Sensor Surface,” Langmuir, 20:6808-6817 (2004), which are hereby incorporated by reference in their entirety.

A wide variety of organic and inorganic materials, both natural and synthetic, and combinations thereof, may be employed for the substrate, provided only that the substrate does not interfere with production of signal from a marker when using the device of the present invention. Illustrative substrates include polymers (e.g., polyethylene, polypropylene, poly(4-methylbutene), polystyrene, polycarbonate, poly(methyl methacrylate), poly(ethylene terephthalate), nylon, poly(vinyl chloride), and poly(vinyl butyrate)), glass, silicon, ceramics, and the like. In a preferred embodiment, the substrate is poly(methyl methacrylate), e.g., Plexiglass or Lucite.

Hydrophilic polymers such as poly(ethylene glycol) or poly(vinylpyrrolidone) can be conjugated to the substrate surface in order to reduce hydrophobic denaturation of proteins. Competing proteins, such as bovine serum albumen, can also be adhered to the substrate surface in order to reduce hydrophobicity.

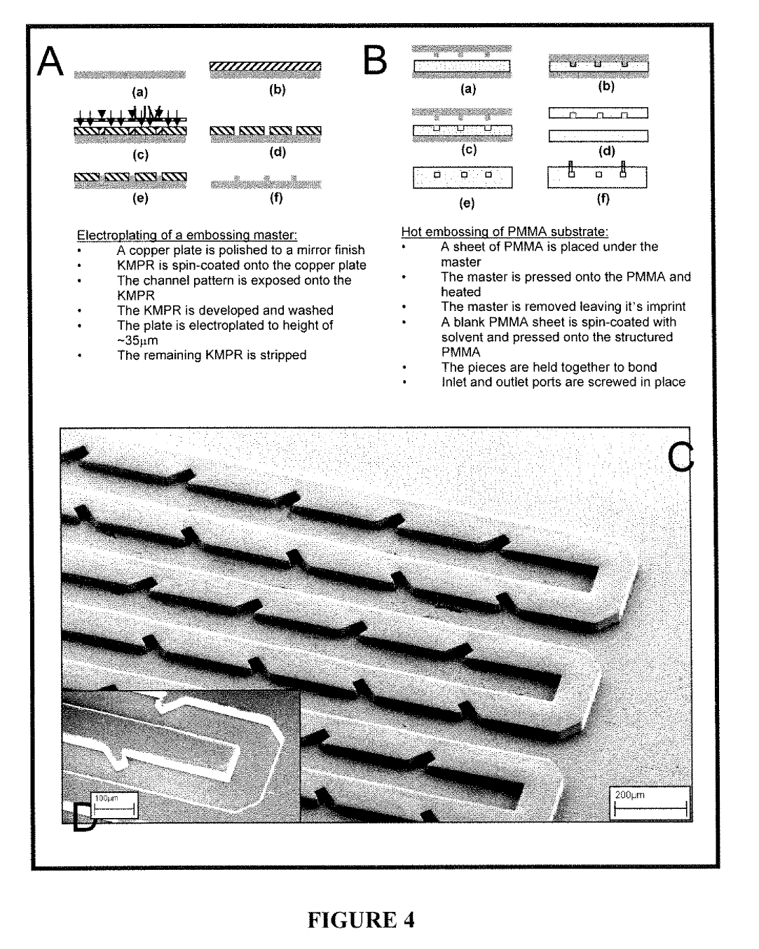

The device of the present invention can be fabricated using any suitable method. An example of a method of making a device in accordance with a preferred embodiment of the present invention is shown in FIGS. 4A-D.

In particular, referring to FIG. 4A, a metal master is made by applying photoresist to a metal plate in a suitable pattern. Suitable photoresists include, but are not limited to, KMPR (MicroChem, Newton, Mass.) and SU-8 (MicroChem, Newton, Mass.). Techniques for application of the photoresist are generally known in the art and include, for example, spin coating and baking. The applied photoresist is then exposed and developed to create the desired pattern, as is known in the art. Additional metal is then deposited to a desired thickness on the areas of the plate not covered by the photoresist. This may be carried out by electroplating, evaporation, or sputtering. The photoresist is then removed leaving the metal master having the desired pattern. Suitable metals include, for example, copper and nickel. Copper is particularly preferred, due to its ease of use, its relatively low cost, and its faster heat transfer properties.

The metal master can then be used to form a microchannel into a polymer substrate, as shown in FIG. 4B. In particular, a first sheet of polymer (i.e., polymer substrate) is placed under the metal master and the master is pressed onto the first polymer sheet and heated. The master is removed leaving its imprint in the first polymer sheet. Another polymer layer is then sealed on top of the first polymer sheet, forming an enclosed microchannel. Preferably, the layers are sealed together by solvent-assisted thermal bonding. Inlet and outlet ports can then be formed in the appropriate positions. Examples of an electroplated copper master and formed polymer substrate produced in accordance with the method shown in FIGS. 4A-B are shown in FIGS. 4C-D.

Although a metal master is used in the embodiment shown in FIGS. 4A-B, other materials may be used, such as silicon. Silicon embossing masters can be prepared using standard photolithographic methods followed by a reactive ion etching process such as the “Bosch” process (Esch et al., “Influence of Master Fabrication Techniques on the Characteristics of Embossed Microfluidic Channels,” Lab Chip, 3:121-127 (2003); Zhao et al., “Fabrication of High-Aspect-Ratio Polymer-Based Electrostatic Comb Drives Using the Hot Embossing Technique,” Journal of Micromechanics and Microengineering, 13:430-435 (2003), which are hereby incorporated by reference in their entirety), as is known in the art.

In one embodiment, the device is used in electrochemical detection assays, as described below. In this embodiment, the device can further include an electrochemical detection device proximate the detection region for determining the presence or amount of analyte in a sample. In particular, the electrochemical device can include one or more electrodes disposed on the device proximate the detection region such that the one or more electrodes are capable of detecting a change in electrical current within the detection region.

The electrode(s) can be made of any suitable material, including, but not limited to, gold, silver, carbon, and platinum. A preferred material is gold.

A preferred electrode is an IDUA shaped by laser ablation from gold evaporated on a polymer surface. In another preferred embodiment, two parallel gold plates are used. In this embodiment, a gold electrode is evaporated on both the top and bottom of the detection region of the device, preferably with a gap of 10 μm or less.

The electrode can be attached to the device using methods that will be apparent to the skilled artisan. In a preferred embodiment, the electrode is attached to the device by an adhesive layer, preferably a thiol terminated molecule. Suitable adhesives include, for example, organosilanes, such as cystamine and mercapto-propyl-tri-methoxy-silane (see description below). Chromium and titanium can also be used as an inorganic adhesive layer.

In another embodiment, the device can be used in optical detection assays, as described below. In this embodiment, the device can further include an optical detection device positioned proximate the detection region for determining the presence or amount of analyte in a sample. Suitable optical detection devices include, but are not limited to, the unaided eye, reflectometers, fluorimeters, and spectrophotometers.

The present invention also relates to a method of detecting or quantifying an analyte in a test sample. This method involves providing a device including a substrate defining one or more microchannels and having a reaction region in a first portion of the one or more microchannels; and a detection region in fluid communication with the reaction region. A test sample potentially comprising a target analyte is introduced into the reaction region, under conditions effective to permit binding between a first binding element present within the reaction region and a first portion of the analyte. The method further includes providing a second binding element selected to immobilize the analyte within the detection region, wherein the second binding element is capable of binding with a second portion of the analyte or a portion of the first binding element and contacting the test sample with the detection region, under conditions effective to immobilize the analyte within the detection region. One or more reporter complexes are provided under conditions effective to permit binding between the reporter complexes and a third portion of the analyte or a portion of the first binding element or a portion of the second binding element. The method further includes detecting any reporter complexes bound to the analyte or first binding element or second binding element in the detection region, and correlating the presence or quantity of bound reporter complexes to the presence or quantity of analyte in the test sample.

A further embodiment of the present invention relates to a method of detecting or quantifying an analyte in a test sample. This method involves providing a device including a substrate defining one or more microchannels and having a reaction region in a first portion of the one or more microchannels and a detection region in fluid communication with the reaction region. A test sample potentially comprising a target analyte is introduced into the reaction region. The method further includes providing one or more reporter complexes, wherein the reporter complexes comprise a first binding element and a marker, under conditions effective to permit binding between the first binding element and a first portion of the analyte and providing a second binding element selected to immobilize the analyte within the detection region, under conditions effective to permit binding between the second binding element and a second portion of the analyte or a portion of the one or more reporter complexes. In addition, the method includes contacting the test sample and the one or more reporter complexes with the detection region, under conditions effective to immobilize the analyte within the detection region, detecting any reporter complexes bound to the analyte in the detection region, and correlating the presence or quantity of bound reporter complexes to the presence or quantity of analyte in the test sample.

Suitable first and second binding elements are described above.

The methods according to these aspects of the present invention may include various steps in which unbound components are removed from the device. This can be carried out by any method suitable in the art, e.g., by rinsing the channel with a suitable buffer.

In one embodiment of the present invention, the analyte is a target nucleic acid molecule and the reaction region is used for both isolation and amplification of the target nucleic acid molecule. Suitable amplification techniques include polymerase chain reaction, ligase chain reaction, and Nucleic Acid Sequence Based Amplification (NASBA) (see Kievits et al., “NASBA Isothermal Enzymatic in vitro Nucleic Acid Amplification Optimized for the Diagnosis of HIV-1 Infection” J. of Virological Methods 35:273-286 (1991), which is hereby incorporated by reference in its entirety). NASBA, marketed by Organon-Teknika, is a preferred amplification technique when determining information regarding the presence or concentration of viable organisms in a sample. However, the target nucleic acid need not be amplified in accordance with the present invention. Alternatively, target nucleic acid may be amplified prior to introducing into the reaction region.

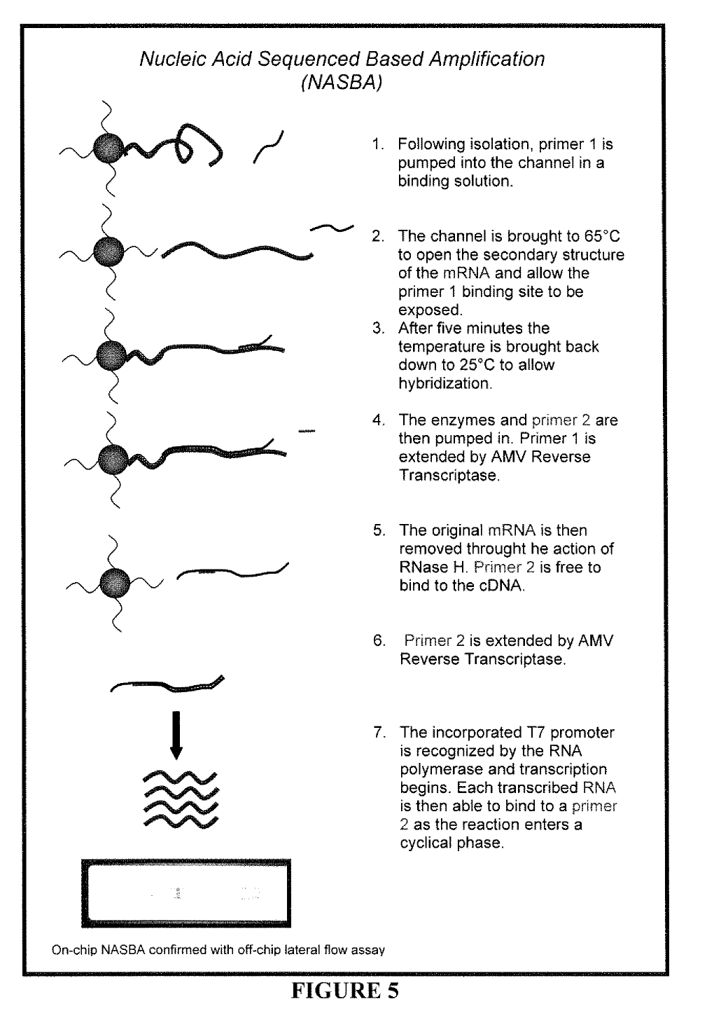

Thus, in a preferred embodiment, the method according to this aspect of the present invention includes introducing primers for NASBA, which is an isothermal RNA amplification reaction used to amplify any target RNA molecules that are bound to nucleic acid capture probes (i.e., first binding element) in the reaction region. Suitable primers can be designed by the skilled artisan based on the nucleic acid sequence of the target RNA molecule. An example of NASBA is shown in FIG. 5. In particular, following binding between the oligonucleotide capture probes (first binding material) and a first portion of the RNA target, the first nucleic acid primer is introduced in a binding solution. The reaction region is brought to the appropriate temperature (e.g., 65° C.) to open the secondary structure of the target RNA and allow the binding site for the first primer to be exposed. After sufficient time, the temperature is brought back down to allow hybridization. Enzymes and the second nucleic acid primer are then introduced and the first primer is extended by AMV Reverse Transcriptase. The original RNA is then removed through the action of RNase H. The second primer is free to bind to the cDNA. The second primer is extended by AMV Reverse Transcriptase. The incorporated T7 promoter is recognized by the RNA polymerase and transcription begins. Each transcribed RNA is then able to bind to the second primer as the reaction enters a cyclical phase.

In this embodiment, the detection region of the device is modified with detection probes specific to the amplified RNA molecules. In one embodiment, the detection probes are immobilized in the detection region.

The methods of the present invention also include introducing reporter complexes into the reaction and/or detection regions of the device. The reporter complexes include a binding element specific for the analyte or a portion of the first binding element and a marker. In one embodiment, the reporter complexes include a third binding element specific for a separate portion of the analyte. In another embodiment, the reporter complexes include the first binding element and a marker. The reporter complexes are allowed to bind to the analyte (or amplified RNA molecules), and unbound reporter complexes are removed. The reporter complexes that remain bound are detected using techniques that are suitable for the particular reporter complexes used, and the presence of the reporter complex is correlated to the presence or quantity of the analyte present in the test sample. The reporter complexes may also include first and second portions, the first portion being provided in the reaction region and the second portion being provided in the detection region of the device.

Any suitable reporter complex can be used in the method of the present invention. Such reporter complexes include a binding element specific for the analyte, e.g., DNA or peptide nucleic acid (PNA) sequences specific for target nucleic acids or, in the embodiment described above, the amplified RNA molecule.

The reporter complex further includes a marker for detection. In a preferred embodiment, the marker includes a particle and a detectable label. Suitable labels include, for example, fluorescent labels, biologically-active labels, radionucleotides, radioactive labels, nuclear magnetic resonance active labels, luminescent labels, chemiluminescent moieties, magnetic particles, chromophore labels, and the like. Preferred labels for electrochemical detection include potassium ferri/ferro cyanide. Preferred labels for fluorescence detection include fluorescein and sulforhodamine B.

Suitable particles include, but are not limited to, liposomes (the label may be encapsulated within the liposome, in the bilayer, or attached to the liposome membrane surface), latex beads, gold particles, silica particles, dendrimers, quantum dots, magnetic beads (e.g., antibody-tagged magnetic beads and nucleic acid probe-tagged magnetic beads), or any other particle suitable for derivatization.

In a preferred embodiment, the reporter complex includes a liposome to which one or more binding elements are attached. In a most preferred embodiment, the reporter complex comprises a liposome encapsulating a label. The one or more binding elements may be conjugated to a liposome surface through coupling groups, as described above. The one or more binding elements must be bound to the liposome or other particle so as to present a portion that may be recognized by the analyte or first binding material.

The use of liposomes as described in the present application provides several advantages over traditional signal production systems employing, for example, enzymes. These advantages include increased signal intensity, shelf stability, and instantaneous release of signal-producing markers, as described in Siebert et al., Analytica Chimica Acta 282:297-305 (1993); Yap et al., Analytical Chemistry 63:2007 (1991); Plant et al., Analytical Biochemistry 176:420-426 (1989); Locascio-Brown et al., Analytical Chemistry 62:2587-2593 (1990); and Durst et al., Eds., Flow Injection Analysis Based on Enzymes or Antibodies, vol. 14, VCH, Weinheim (1990), each of which is hereby incorporated by reference in its entirety.

Liposomes can be prepared from a wide variety of lipids, including phospholipids, glycolipids, steroids, relatively long chain alkyl esters; e.g., alkyl phosphates, fatty acid esters; e.g. lecithin, fatty amines, and the like. A mixture of fatty materials may be employed, such as a combination of neutral steroid, a charge amphiphile, and a phospholipid. Illustrative examples of phospholipids include lecithin, sphingomyelin, and dipalmitoylphosphatidylcholine, etc. Representative steroids include cholesterol, chlorestanol, lanosterol, and the like. Representative charge amphiphilic compounds generally contain from 12 to 30 carbon atoms. Mono- or dialkyl phosphate esters, or alkylamines; e.g. dicetyl phosphate, stearyl amine, hexadecyl amine, dilaurylphosphate, and the like are representative.

The liposome sacs are prepared in aqueous solution containing the label whereby the sacs will include the label in their interiors. They may also contain the label bound to the exterior layer or embedded within the lipid layer. The liposome sacs may be prepared by vigorous agitation in the solution, followed by removal of the unencapsulated label. Further details with respect to the preparation of liposomes are set forth in U.S. Pat. No. 4,342,826 and PCT International Publication No. WO 80/01515, both of which are hereby incorporated by reference in their entirety.

As described above, the methods and test devices of the present invention may be modified to use an electrochemical marker. In the electrochemical detection method of the invention, an electroactive species, such as ferrocyanide, is encapsulated in the marker, e.g., liposomes. Electrodes are printed onto the substrate, or the substrate is placed in contact with reusable electrodes, such as an interdigitated ultramicroelectrode array (IDUA). After lysis of the liposomes, the quantity of the electroactive species is determined.

Suitable electrochemical markers, as well as methods for selecting them and using them are disclosed, for example, in U.S. Pat. No. 5,789,154 to Durst et al., U.S. Pat. No. 5,756,362 to Durst et al., U.S. Pat. No. 5,753,519 to Durst et al., U.S. Pat. No. 5,958,791 to Roberts et al., U.S. Pat. No. 6,086,748 to Durst et al., U.S. Pat. No. 6,248,956 to Durst et al., U.S. Pat. No. 6,159,745 to Roberts et al., U.S. Pat. No. 6,358,752 to Roberts et al., and co-pending U.S. patent application Ser. No. 10/264,159, filed Oct. 2, 2002, which are hereby incorporated by reference in their entirety. Briefly, the test device may designed for amperometric detection or quantification of an electroactive marker. In this embodiment, the test device includes a working electrode portion(s), a reference electrode portion(s), and a counter electrode portion(s) on the substrate of the test device. The working electrode portion(s), reference electrode portion(s), and counter electrode portion(s) are each adapted for electrical connection to one another via connections to a potentiostat. Alternatively, the test device may be designed for potentiometric detection or quantification of an electroactive marker. In this embodiment, the test device includes an indicator electrode portion(s) and a reference electrode portion(s) on the substrate of the test device. The indicator electrode portions and reference electrode portions are adapted for electrical connection to potentiometers. In another embodiment, the test device may include an IDUA positioned to induce redox cycling of an electroactive marker released from liposomes upon lysis of the liposomes.

Suitable electroactive markers are those which are electrochemically active but will not degrade the particles (e.g., liposomes) or otherwise leach out of the particles. They include metal ions, organic compounds such as quinones, phenols, and NADH, and organometallic compounds such as derivatized ferrocenes. In one embodiment, the electrochemical marker is a reversible redox couple. A reversible redox couple consists of chemical species for which the heterogeneous electron transfer rate is rapid and the redox reaction exhibits minimal overpotential. Suitable examples of a reversible redox couple include, but are not limited to, ferrocene derivatives, ferrocinium derivatives, mixtures of ferrocene derivatives and ferrocinium derivatives, cupric chloride, cuprous chloride, mixtures of cupric chloride and cuprous chloride, ruthenium-tris-bipyridine, potassium ferrohexacyanide, potassium ferrihexacyanide, and mixtures of potassium ferrohexacyanide and potassium ferrihexacyanide. Preferably, the electrochemical marker is encapsulated within a liposome, in the bilayer, or attached to a liposome membrane surface.

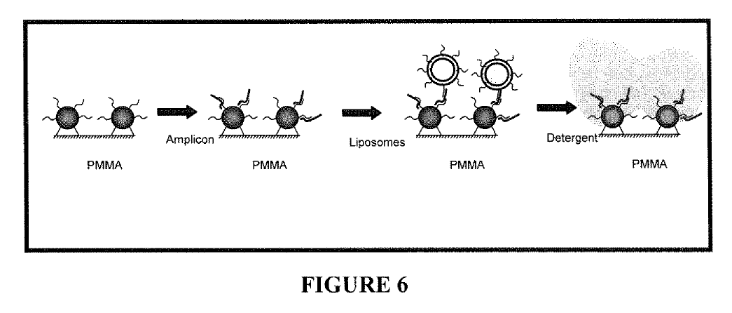

As described above, the methods and test devices of the present invention may also be modified to use an optical marker. An example of a detection method in accordance with this aspect of the present invention is shown in FIG. 6. In particular, the detection region of the device is modified with a detection probe specific to the amplified RNA (amplicon). The detection probe is immobilized using a dendrimer as a linker layer. Dye encapsulating liposomes (i.e., reporter complexes) are then introduced into the detection region to mark the bound amplified RNA. The introduction of a detergent then lyses the liposomes, thereby releasing the dye and increasing the fluorescence. The dye can then be detected using, for example, a CCD camera, as is known in the art.

Although the present invention describes a sandwich assay, competition assays are also contemplated. As will be apparent to the skilled artisan, the method of the present invention can be modified for competition assays by using reporter complexes that are configured to bind competitively to the second binding element, rather than to the analyte.

The device and method of the present invention can be used to evaluate any test sample that potentially contains a target analyte. The test sample may be derived from a wide variety of sources, such as environmental samples (e.g., water (waste water, natural waters), chemical processing streams, air, and soil extracts), a food sample, a beverage sample, or a biological sample (e.g., blood, serum, saliva, sweat, plasma, urine, tear fluid, and spinal fluid) that potentially contains a pathogen (e.g., virus, parasite, fungus, bacteria, etc.) or other analyte. In this embodiment, the target analyte is any analyte that is specific to the pathogen. The device and methods of the present invention have relevant uses in healthcare, food and beverage analysis, and environmental analysis.

In carrying out the methods of the invention, the sample suspected of containing the analyte may be combined with one or more of the reporter complex(es), first binding element, and second binding element (and other desired components) in an electrolytic aqueous medium to form an aqueous test mixture or solution. Various addenda may be added to adjust the properties of the test mixture depending upon the properties of the other components of the device, as well as on those of the marker complexes, conjugates, or the analyte itself. Examples of solution addenda which may be incorporated into test, control, or carrier solutions or mixtures in accordance with the invention include buffers, for example, pH and ionic strength, sample or analyte solubilizing agents, such as, for example, nonpolar solvents, and high molecular weight polymers such as Ficoll®, a nonionic synthetic polymer of sucrose, available from Pharmacia, and dextran.

The order of addition of the test sample (suspected of containing the analyte), the reporter complex(es), the first binding element, and/or the second binding element to one another is not critical.

The method of addition of the test sample, the marker complex(es), the first binding element, and/or the second binding element to the device is also not critical. For example, sample/test mixture can be deposited in a well on the device and pulled into the device by a syringe. Alternatively, sample/test mixture can be pulled into the device through tubing. In another embodiment, the sample/test mixture can be pumped directly into the device using positive pressure.

For the most part, relatively short times are involved for the test mixture/sample to traverse the device. Usually, traversal of the test mixture/sample through the microchannel(s) of the device will take at least ten seconds and not more than five minutes, more usually from about one minute to about three minutes. In accordance with the method of the invention, the signal is rapidly, even immediately, detectable.

As hereinabove indicated, the signal producing system includes a marker complex which includes a binding element and a marker, e.g., a label within the interior of derivatized liposomes. Suitable markers include fluorescent dyes, visible dyes, bio- and chemiluminescent materials, quantum dots, enzymes, enzymatic substrates, radioactive materials, and electroactive markers. When using liposomes in the reporter complex, visible dyes and radioactive materials can be measured without lysis of the liposomes. Lysis of the liposomes in the device and methods of the present invention may be accomplished by applying a liposome lysing agent to the substrate, for example, in the detection region. Suitable liposome lysing materials include surfactants such as octylglucopyranoside, sodium dioxycholate, sodium dodecylsulfate, saponin, polyoxyethylenesorbitan monolaurate sold by Sigma under the trademark Tween-20, and a non-ionic surfactant sold by Sigma under the trademark Triton X-100, which is t-octylphenoxypolyethoxyethanol. Octylglucopyranoside is a preferred lysing agent for many assays, because it lyses liposomes rapidly and does not appear to interfere with signal measurement. Alternatively, complement lysis of liposomes may be employed, or the liposomes can be ruptured with electrical, optical, thermal, or other physical means.

As will be appreciated by the skilled artisan, the method of the present invention may be used qualitatively (e.g., determining whether or not analyte is present), semi-quantitatively, and quantitatively. For quantitative measurements, the amount of analyte in the sample can be roughly estimated based on the amount of bound reporter complex. For example, in embodiments using fluorescence reporters, the intensity of the reporter signal is roughly proportional (directly or inversely, depending upon whether a sandwich or competition assay is used) to the amount of analyte in the test sample.

A qualitative or semi-quantitative measurement of the presence or amount of an analyte of interest may be made with the unaided eye when visible dyes are used as the marker. The intensity of the color may be visually compared with a series of reference standards, such as in a color chart, for a semi-quantitative measurement. The preparation of suitable standards and/or standard curves (the term “standard curve” is used in a generic sense to include a color chart) is deemed to be within the scope of those skilled in the art from the teachings herein. Alternatively, when greater precision is desired, or when the marker used necessitates instrumental analysis, the intensity of the marker may be measured directly on the substrate using a quantitative instrument such as a reflectometer, fluorimeter, spectrophotometer, electroanalyzer, etc.

The solvent for the test mixture will normally be an aqueous medium, which may be up to about 40 weight percent of other polar solvents, particularly solvents having from 1 to 6, more usually of from 1 to 4, carbon atoms, including alcohols, formamide, dimethylformamide and dimethylsulfoxide, dioxane, and the like. Usually, the cosolvents will be present in less than about 30-40 weight percent. Under some circumstances, depending on the nature of the sample, some or all of the aqueous medium could be provided by the sample itself.

The pH for the medium will usually be in the range of 4-10, usually 5-9, and preferably in the range of about 6-8. The pH is chosen to maintain a significant level of binding affinity of the binding members and optimal generation of signal by the signal producing system (i.e., reporter complex(es)). Various buffers may be used to achieve the desired pH and maintain the pH during the assay. Illustrative buffers include borate, phosphate, carbonate, tris, barbital, and the like. The particular buffer employed is usually not critical, but in individual assays, one buffer may be preferred over another. For nucleic acid analytes, it is necessary to choose suitable buffers. Such buffers include SSC, sodium chloride, sodium citrate buffer, and SSPE (sodium chloride, sodium phosphate, EDTA).

The concentration of electrolytes in the medium will usually be adjusted to achieve isotonicity or equi-osmolality (or up to about 50 to about 100 mmol/kg hypertonic) with the solution in the interior of liposomes to prevent their crenation or swelling.

With some increased complexity of the excitation waveform applied by an electroanalyzer, electrochemical measurement in accordance with the invention may also be carried out using stripping voltammetry, employing, for example, liposome encapsulated metal ions for detection and measurement.

Moderate, and desirably substantially constant, temperatures are normally employed for carrying out the assay. The temperatures for the assay and production of a detectable signal will generally be in the range of about 4-65° C., more usually in the range of about 20-38° C., and frequently, will be about 15-45° C.

The concentration, in the liquid sample, of analyte which may be assayed will generally vary from about 10⁻³ to about 10⁻²⁰M, more usually from about 10⁻⁵ to 10⁻¹⁵M. Considerations such as the concentration of the analyte of interest and the protocol will normally determine the concentration of the other reagents.

It will be understood by the skilled artisan that the device and methods of the present invention can be modified to detect more than one target analyte, such as toxic chemicals or pathogens, or screen for one or more of a plurality of analytes. In this embodiment, multiple first and second binding elements each specific for a particular analyte, and multiple reporter complexes, each specific for the particular target analyte, are used. Where multiple markers are used, each marker may be the same or different. In the case that each marker is different, each reporter complex is specific for a particular target analyte. Each analyte may be determined by assignment of each conjugate/analyte to its own measurement portion within the detection region for concentration and measurement. Alternatively, the conjugate of each of the analytes to be determined in this embodiment of the invention, may include a marker which is detectable distinctly from the other markers. With different encapsulated dyes (e.g., fluorescent dyes) or quantum dots, the results of the assay can be “color coded.” In particular, a multi-wavelength detector can be used in a capture portion.

In this embodiment, the reaction region of the device may include a plurality of microchannels, each for a particular target analyte. Similarly, the detection region of the device may include a plurality of microchannels, each for a particular analyte. Alternatively, a single microchannel may be used in the reaction and detection regions to detect more than one target analyte.

As a matter of convenience, the present device can be provided in a kit in packaged combination with predetermined amounts of reagents for use in assaying for an analyte or a plurality of analytes. Aside from the substrate having one or more microchannels, substrate having one or more microchannels and the second binding element immobilized thereto, and substrate having one or more microchannels and the first and second binding elements immobilized thereto, first binding element, second binding element, and/or reporter complex, as well as other additives such as ancillary reagents may be included, for example, stabilizers, buffers, and the like. The relative amounts of the various reagents may be varied widely, to provide for concentration in solution of the reagents which substantially optimizes the sensitivity of the assay. The reagents can be provided as dry powders, usually lyophilized, including excipients, which on dissolution will provide for a reagent solution having the appropriate concentrations for performing the assay. The kit or package may include other components such as standards of the analyte or analytes (analyte samples having known concentrations of the analyte).

The present invention is applicable to procedures and products for determining a wide variety of analytes. As representative examples of types of analytes, there may be mentioned: environmental and food contaminants, including pesticides and toxic industrial chemicals; drugs, including therapeutic drugs and drugs of abuse; hormones, vitamins, proteins, including enzymes, receptors, and antibodies of all classes; prions; peptides; steroids; bacteria; fungi; viruses; parasites; components or products of bacteria, fungi, viruses, or parasites; aptamers; allergens of all types; products or components of normal or malignant cells; etc. As particular examples, there may be mentioned C. parvum, T₄; T₃; digoxin; hCG; insulin; theophylline; leutinizing hormones; and organisms causing or associated with various disease states, such as streptococcus pyogenes (group A), Herpes Simplex I and II, cytomegalovirus, chlamydiae, etc. The invention may also be used to determine relative antibody affinities, and for relative nucleic acid hybridization experiments, restriction enzyme assay with nucleic acids, binding of proteins or other material to nucleic acids, and detection of any nucleic acid sequence in any organism, i.e., prokaryotes and eukaryotes.

The methods according to the present invention allow for assays to be performed in a number of different formats, as described in detail herein. Some preferred formats include: (1) wherein the first binding element is bound in the reaction region for amplification of a target nucleic acid and the second binding element is immobilized in the detection region; (2) wherein the first binding element is a first portion of a capture element that binds to the analyte and is in solution with the test sample in the reaction region and the second binding element is a second portion of the capture element and is provided on a magnetic bead for immobilization in the detection region during detection (and could be added into either the reaction region or the detection region) or is immobilized to a surface of the detection region; (3) wherein reporter complexes, which include the first binding element which binds to the analyte, and a capture element (i.e., the second binding element) including a magnetic bead are mixed in the reaction region with the test sample; (4) wherein reporter complexes, which include the first binding element which binds to the analyte, and test sample are mixed in the reaction region, with a capture element (i.e., second binding element) immobilized in the detection region (either attached to a surface of the detection region or, when attached to magnetic beads, introduced and held in detection region with a magnet); and (5) wherein a capture element (i.e., second binding element) including a magnetic bead is mixed with test sample in the reaction region and reporter complexes, which include the first binding element which binds to the analyte, are added in the detection region

Yet another embodiment of the present invention relates to a method for coating a polymer with a gold layer. This method involves providing a polymer having at least a portion of a surface having a plurality of carboxylic acids, conjugating a heterobifunctional molecule containing an amino group and a thiol group to the surface under conditions effective to produce a thiolated surface, and adhering a gold layer to the thiolated surface.

Suitable polymers include, but are not limited to, polyethylene, polypropylene, poly(4-methylbutene), polystyrene, polycarbonate, polymethacrylate, poly(ethylene terephthalate), nylon, poly(vinyl chloride), poly(vinyl butyrate), and the like. In a preferred embodiment, the polymer is poly(methyl methacrylate), e.g., Plexiglass or Lucite.

Suitable heterobifunctional molecules in accordance with the present invention include, but are not limited to, cystamine, cystine, and 3-(2-pyridyldithio) propiony hydaride (PDPH).

In accordance with this embodiment of the present invention, providing includes treating at least a portion of a surface of the polymer under conditions effective to form a plurality of carboxylic acids on the surface. Treating may be achieved by methods known to those of skill in the art, including UV irradiation, O₃ exposure, UV/O₃ combination, H₂SO₄ hydrolysis, and corona discharge. In a preferred embodiment, treating comprises UV irradiation. In another preferred embodiment, treating results in a carboxylic acid density of from about 0.1 nmol/cm² to about 100 nmol/cm², preferably from about 1 nmol/cm² to about 10 nmol/cm², on the surface.

Suitable methods for conjugating including, but are not limited to, water soluble carbodiimide chemistries. Examples of conjugation of primary amines to carboxylic acids using water soluble carbodiimides can be found in Hermanson, Bioconjugate Techniques, Elsevier Science (1996), which is hereby incorporated by reference in its entirety. The gold is then adhered using gold-thiol interactions.

Gold held to the polymer surface via an adhesion layer in accordance with the present invention can be used not only as electrodes for assays as described herein, but can be used for general gold coating of polymer substrates. This gold could be used as electrodes for any electrochemical assay, it could also be used as an immobilization layer for surface plasmon resonance (SPR), cantilevers, evanescent wave detection systems, acoustic wave, and other acoustic and mechanical detection systems.

The present invention may be further illustrated by reference to the following examples.

### EXAMPLES

### Example 1

### Micro-Total Analysis System Using Electrochemical Detection

A micro-Total Analysis System for the detection of RNA derived from cells or viruses is presented. As an initial model, analyte Cryptosporidium parvum was chosen. A poly(methyl methacrylate) (“PMMA”) biosensor was designed and fabricated which has the ability of detecting less than five C. parvum oocysts. Current EPA water standards incorporate labor-intensive microscopy which limits sample throughput. An automated and inexpensive biosensor could increase sample loads without much effect on labor.

Cryptosporidium parvum

Lysed C. parvum oocysts (Iowa isolate) were supplied by Wisconsin State Lab of Hygiene (Madison, Wis.). The oocysts were counted using flow cytometry and then lysed in a lysis binding buffer (100 mM Tris-HCl, pH 7.5, 500 mM LiCl, 10 mM EDTA, 1% LiDS, 5 mM DTT) using five freeze-thaw cycles. Oligo (dT)₂₅ beads (Dynal, Oslo, Norway) were placed in the lysed sample and then allowed to hybridize for five minutes with gentle shaking. The sample was then aspirated on a magnetic stand. The sample was then rinsed twice with wash buffer A (10 mM Tris-HCl, pH 7.5, 0.15 mM LiCl, 1 mM EDTA, 0.1% LiDS) and twice with wash buffer B (10 mM Tris-HCl, pH 7.5, 0.15 mM LiCl, 1 mM EDTA). The washed beads were then resuspended in 5 μL of nuclease-free water and the mRNA was amplified using the NuliSens NASBA kit (bioMerieux, Durham, N.C.). The NASBA primers and protocol have previously been shown to yield high amplification efficiency (Esch et al., Anal. Chem., 73:3162-3167 (2001), which is hereby incorporated by reference in its entirety). The NASBA amplicon was then stored at −80° C. until needed.

### Device Design

The device contained an hybridization/amplification chamber (i.e., reaction region) and a detection channel (i.e., detection region). A sawtooth mixer (Nichols et al., Lab Chip, 6:242-246 (2006), which is hereby incorporated by reference in its entirety) was incorporated into the hybridization/amplification chamber to aid in mixing during loading. The detection channel contained no sawtooth structures but contained the IDUA. The electrode was designed with a 5 μm gap between the fingers and the finger width of 10 μm. The finger width was wider than previously used in order to increase the surface area of the electrode to maintain adhesion during use (Goral et al., Lab Chip, 6:414-421 (2006), which is hereby incorporated by reference in its entirety). A second inlet channel was designed to insert the detergent for induced lysis of liposomes to enable the amperometric detection of ferro/ferrihexacyanide released from the liposomes.

### IDUA Formation

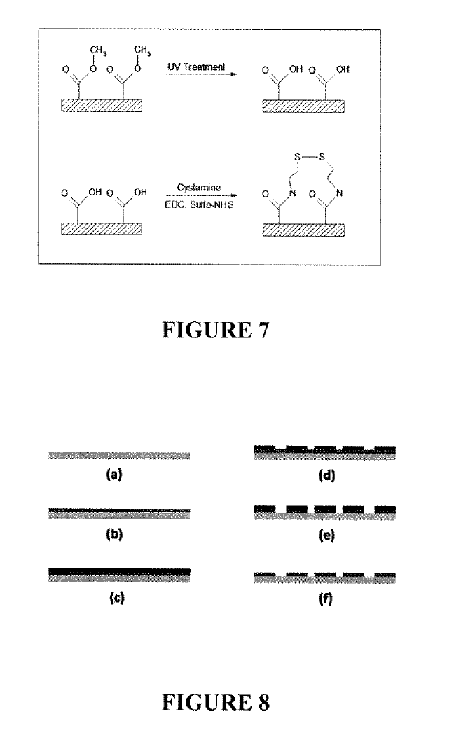

The PMMA (Lucite International, Southampton, UK) was cut into 50 mm×50 mm pieces and then sonicated in 50% 2-propanol for ten minutes prior to use. The adhesion of gold electrodes on the PMMA surface was accomplished by thiolating the PMMA surface (FIG. 7). The surface thiolation required an initial UV treatment to induce carboxyl formation. Cystamine could then be conjugated to the carboxylated surface using water soluble carbodiimide chemistry.

A UV/ozone stripper (SAMCO International, Inc., Sunnyvale, Calif.) was used for the oxidation. Initially, the effect of UV, ozone and UV/ozone were evaluated individually for a 10 minute treatment (see Example 3, below). The PMMA was treated with UV (10 mW/cm² at 254 nm) for eight minutes.

The PMMA pieces were then placed in agitated DI water for 30 minutes for a post exposure rinse before being dried with nitrogen. For the surface conjugation, 1 mL of a 0.05 M MES, pH 6.0 containing 300 mM EDC (G-Biosciences, St. Louis, Mo.) and 300 mM sulfo-NHS (G-Biosciences, St. Louis, Mo.) was placed in the middle of a 4-inch petri dish. The PMMA was then placed onto the solution with the UV-exposed side in contact with the liquid. After 25 minutes, the PMMA was rinsed and then placed face down in another petri dish with 1 mL of 0.05 M sodium carbonate buffer, pH 9 containing 300 mM cystamine (Alfa Aesar, Ward Hill, Mass.). Following a three hour conjugation period, the PMMA was again rinsed with DI water and dried with nitrogen.

The gold electrodes were formed on the thiolated PMMA surface using standard photolithographic methods followed by a gold etch (FIG. 8). The thiol-functionalized PMMA was coated with gold using a CHA Mark 50 evaporator (CHA Industries, Freemont, Calif.). Briefly, the gold was deposited at 0.25 nm/s for a total thickness of 200 nm. Following evaporation, the positive photoresist S1827 (Shipley Co., Marlborough, Mass.) was then spun onto the gold-coated PMMA at 3000 rpm for 30 seconds. The piece was baked in a 90° C. oven for 20 minutes to allow the solvent to evaporate. Following baking, the photoresist was UV exposed for five seconds through a mask containing the electrode pattern using a contact aligner (Hybrid Technology Group). The piece was then placed back in the 90° C. oven for an additional five minutes. Following the post-exposure bake, the resist was developed in MIF-321 (Shipley Co., Marlborough, Mass.) for two minutes and then rinsed with DI water before being dried with N₂. The electrode was formed by submerging the piece in gold etchant (Transene, Co. Inc., Danvers, Mass.) for approximately three minutes. The remaining surface was then again washed with deionized water and then dried with nitrogen.

The PMMA and remaining photoresist were then exposed to UV for eight minutes. This step served to both modify the PMMA for bonding as well as expose the remaining photoresist to allow for its removal in situ following bonding.

### Surface Modification Validation

Toluidine blue O (TBO) has previously been used to quantify carboxylic acid on a polymer surface (Goddard et al., J. Food Sci., 72:E36-E41 (2007); Kang et al., Macromolecules, 29:6872-6879 (1996), which are hereby incorporated by reference in their entirety). TBO is a dye which adsorbs to carboxyls in a low pH solution and then desorbs at a higher pH. This assay was performed on 0.6 cm diameter PMMA discs from the same stock sheet as the PMMA to be used for electrodes deposition. The dye assay was performed on the PMMA prior to being UV treated, following UV treatment, and following cystamine conjugation. The PMMA discs were agitated in a 0.5 mM TBO solution, pH 10, for four hours in order to allow for dye adsorption to the carboxyls. Following the adsorption step, the discs were rinsed in pH 10 deionized water to remove free dye. The discs were then vortexed in 200 μL of 50% weight acetic acid for ten seconds to desorb the dye. The concentration of dye in the desorbing solution was determined by absorbance at 633 nm and comparison to a standard curve of TBO in 50% weight acetic acid.

In addition to the dye assay, the water contact angle was measured on the PMMA. The unmodified PMMA was compared to the UV treated PMMA as well as a UV treated PMMA which was rinsed in deionized water for one hour. The PMMA pieces were placed horizontally on a VCA Optima XE goniometer (AST Products Inc., Boston, Mass.). Two microliters of distilled water were then deposited on the surface and the droplet image was immediately captured. The contact angle was determined using the installed software.

### Device Fabrication

The PMMA channels were formed using hot embossing. The copper hot embossing master was fabricated as previously herein. The channel design included five lengths joined in a serpentine, all of which incorporate a sawtooth micromixer. The sawtooth micromixer was previously shown to enhance mixing of tandem fluids (Nichols et al., Lab Chip, 6:242-246 (2006), which is hereby incorporated by reference in its entirety). The mixer was incorporated to aid in the hybridization of the C. parvum amplicon to the superparamagnetic capture beads and liposomes.

The PMMA channel was hot embossed using a Fortin Hot Press (Forth CRC Prepreg). The PMMA was sandwiched between the copper master and a blank copper plate and then pressed at 130° C. with a force of 1350 N for five minutes. For de-embossing, the newly structured PMMA and copper master were allowed to cool for approximately one minute at room temperature. The PMMA was then manually removed from the copper master. Following de-embossing, the inlet and outlet holes were drilled using 0.8 mm steel bits. The PMMA was then again rinsed and dried with deionized water and N₂, respectively.

The channel structured PMMA and electrode modified PMMA pieces were bonded using UV-assisted thermal bonding (Tsao et al., Lab Chip, 7:499-505 (2007), which is hereby incorporated by reference in its entirety). This process used photochemically induced main chain scission to lower the glass transition temperature of the PMMA surface. The extent and depth of the scission can be controlled with UV duration. A bonding temperature higher than that of the surface, but lower than that of the bulk PMMA can then be selected. This process allows for a solvent-free bonding with no thermal distortion of the PMMA channel. The PMMA adhering the electrode was not affected due to the masking by the electrode itself.

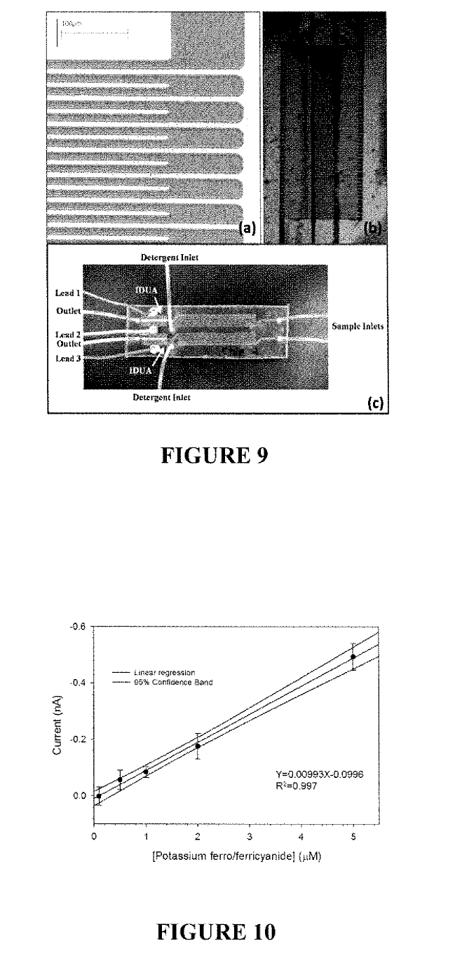

The two PMMA pieces, having been UV functionalized for eight minutes, were aligned and placed in a pneumatic press at 80° C. with a force of 1350 N for three minutes. The bonded pieces were then removed and the inlet and outlet tubing was glued in place using a cyanoacrylate based adhesive (Henkel Consumer Adhesives, Inc., Avon, Ohio) (FIGS. 9A-C). The remaining resist in the channel and contact pads was removed with a five minute treatment in 100 mM NaOH. The leads for the IDUA were coated with silver epoxy (MG Chemicals, Burlington, ON) and adhered to the contact pads.

### Assay

To characterize the IDUA, various concentrations of potassium ferri/ferrohexacyanide were injected into the channel at a flow rate of 5 μL/min. A 400 mV potential was applied across the IDUA and the resulting current was measured on an Epsilon potentiostat (Bioanalytical Systems, Inc., West Lafayette, Ind.).

Liposomes were prepared using reverse phase evaporation as described by Goral et al., Lab Chip, 6:414-421 (2006), which is hereby incorporated by reference in its entirety. The reporter probe, (5′-3′) GTG CAA CTT TAG CTC CAG TT-CHOLESTEROL (SEQ ID NO:1) (Operon, Huntsville, Ala.) was incorporated into the lipid bilayer using a cholesterol tag. Streptavidin coated superparamagnetic beads (1 μm diameter) were conjugated with a biotin-tagged capture probe, (5′-3′) BIOTIN-AGA TTC GAA GAA CTC TGC GC (SEQ ID NO:2) also as described in Goral et al., Lab Chip, 6:414-421 (2006), which is hereby incorporated by reference in its entirety.

For the C. parvum assay, 1 μL of NASBA amplicon was combined with 1 μL of capture beads, 1 μL of liposomes, and 1 μL of a hybridization mixture (50% formamide, 10×SSC, 0.5% ficoll, 0.3 M sucrose, 8% dextran sulfate). The solution was pumped into the channel and allowed to hybridize for 15 minutes. During the hybridization, the solution was pumped in a reciprocating motion (0.5 μL; 1 μL/min.) in order to aid in mixing. Following the hybridization step, a rare earth magnet was placed over the channel in order to hold the beads in place while the sample solution was pumped out and replaced with a washing solution (20% formamide, 4×SSC, 0.2% ficoll, 0.125 M sucrose, 5% dextran sulfate) at 1.5 μL/minute. The magnet was about 1 mm removed from the middle of the channel. The first magnet was then removed and a second rare earth magnet was placed directly over the IDUA. The beads were then washed toward the IDUA again at 1.5 μL/min. for eight minutes. The placement of the second magnet captured the majority of beads directly upstream of the IDUA. The washing step was needed to remove any unhybridized liposomes from the sample area. A detergent, n-Octyl-β-D-glucopyranoside (OG) was then injected at 60 mM in water into the channel at 1 μL/min in order to lyse the liposomes and release the encapsulated potassium ferri/ferrocyanide solution which was in turn detected by the IDUA. The resulting current from the redox reaction was coulometrically measured and recorded. The resulting area under the current/time curve (nA s) was determined to be the assay signal.

### Results and Discussion

Initially the effect of UV, ozone, and the combination of UV and ozone was evaluated in order to determine the optimal method for functionalization (see Example 3 below for a detailed discussion). A 10 minute treatment of each method resulted in much higher surface functionalization for the UV treatment over the ozone and UV/ozone treatments. The water contact angle of unmodified PMMA was found to be 62.5°±0.7°. Immediately following the UV treatment, the water contact angle dropped to 22.9°±0.4°. The effect of UV on PMMA has been found to involve both side chain modification as well as main chain scission (Truckenmuller et al., Microsyst. Technol., 10:372-374 (2004), which is hereby incorporated by reference in its entirety). The scission of the main results in the creation of smaller chains with some of them no longer bound to the surface. Following the rinsing step, which was used to remove the small soluble polymer chains, the contact angle was found to be 48.4°±0.2°.

The TBO dye assay determined that the initial UV treatment provided a carboxylation density of 8 nmol/cm² of carboxyls. Following the conjugation of cystamine the carboxyl density dropped to approximately half. The loss of carboxyls suggests the successful immobilization of cystamine to the PMMA surface. The 50% coupling efficiency is most likely due surface diffusion limitations during the 25 minutes of EDC/NHS activation.

The IDUAs were examined using scanning electron microscopy in order to determine effective etch times (FIG. 9A). The UV-treatment prior to bonding resulted in observable etching between the IDUA fingers and is likely due to PMMA main chain scission (Truckenmuller et al., Microsyst. Technol., 10:372-374 (2004); Ponter et al., Polym. Eng. Sci., 34:1233-1238 (1994), which are hereby incorporated by reference in their entirety).

In fact, the initial bonding procedure, where all photoresist was removed prior to bonding, resulted in significant damage to many of the IDUAs. The yield of working bonded IDUAs was increased by leaving the photoresist on the IDUA during bonding to help protect the gold fingers. The photoresist was removed in situ by pumping 100 mM NaOH through the bonded channel. The NaOH was able to remove the UV-treated photoresist within five minutes.

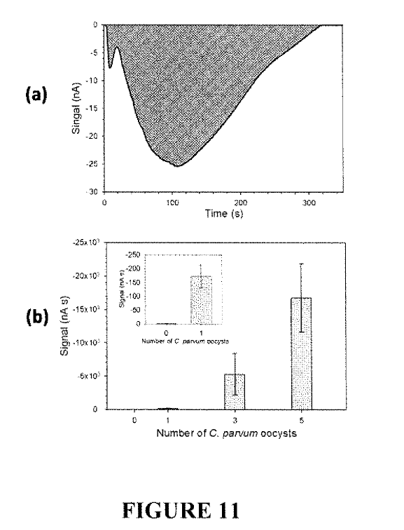

The individual IDUAs were characterized using a dose response to varying potassium ferro/ferricyanide solutions (FIG. 10). As determined earlier, the smaller the gap size the lower the limit of detection (Min et al., Electroanalysis, 16:724-729 (2004), which is hereby incorporated by reference in its entirety). However, the gap size used here on the PMMA substrate was sufficient to provide a low limit of detection of a single oocyst amplicon. The NASBA amplicons from 0, 1, 3 and 5 C. parvum oocysts were analyzed. Amplicons from 1, 3 and 5 oocysts were confirmed positive using a lateral flow assay as previously described (Connelly et al., Anal. Bioanal. Chem., 391:487-495 (2008), which is hereby incorporated by reference in its entirety). The results showed the ability of the IDUA detecting the amplicon from a single C. parvum oocyst (FIG. 11A-B). All oocysts concentrations were statistically distinguishable with a P-value below 0.05 when analyzed with a student t-test. Although peak heights of the individual signals varied, area calculations (FIG. 11A) gave reproducible results. The assays displayed very low backgrounds indicating a low level of non-specific binding of liposomes. This is likely due to negatively charged liposomes used in a modified PMMA channel with a negative surface charge due to surface carboxylic acids.

### CONCLUSION

A PMMA based microfluidic biosensor with electrochemical detection ability has been developed. Although the polymer biosensor was designed to be disposable, the surface modification of the PMMA allowed the IDUA to adhere to the PMMA during repeated use. The use of a liposomal detection system encapsulating potassium ferro/ferricyanide allowed for the detection of amplicon from a single C. parvum oocyst. The gap and the finger width of an IDUA can be further narrowed in order to generate even more sensitive electrochemical transducers for use in biosensing systems.

The UV-assisted thermal bonding proved gentle enough to allow most of the IDUA fingers to remain intact. When solvent bonding was used, the IDUAs were destroyed during bonding. Therefore, UV-assisted thermal bonding was shown to be a preferred technique for bonding PMMA with sensitive surfaces.

The described device was able to detect less than five oocysts in solution within about two hours. The disposable PMMA chip measures less than 2.5 cm×5 cm and can be easily manufactured.

### Example 2

### Micro-Total Analysis System Using Fluorescence Detection

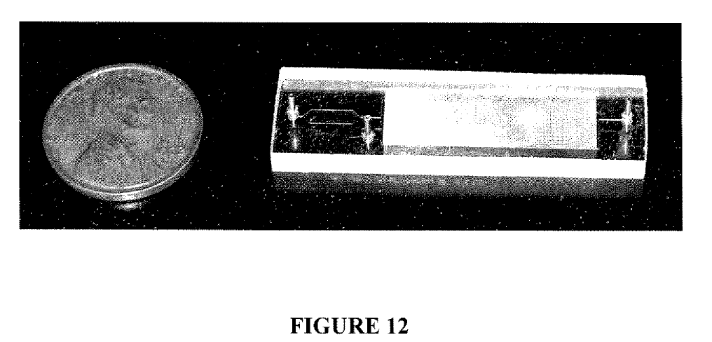

A microfluidic biosensor that isolates mRNA, amplifies the hsp70 gene, and detects an amplicon resulting from less than ten oocysts using fluorescence detection was developed. The present example relates to a biosensor, having a surface functionalized microchannel, fabricated as described in Example 1, except that the system was designed to detect target RNA using fluorescence detection. Fluid flow through the channel network was established by applying a positive pressure at the inlet using a syringe pump (KD Scientific, Inc., Holliston, Mass.) and opening the outlet to atmospheric pressure. The connection between the top of the steel tubing and 500-L Hamilton gastight syringes on the pump was made via Tygon tubing with an inner diameter of 0.5 mm. All channels were prefilled with running buffer (10% formamide, 3×SSC (1×SSC contains 15 mM sodium citrate and 150 mM sodium chloride, pH 7.0), 0.2 M sucrose, 0.2% Ficoll type 400, 0.01% Triton X-100, 10% dextran sulfate) at a slow flow rate of 1 L/minute to prevent the formation of bubbles, In a microcentrifuge tube, 1 L of a hybridization solution (master mix) in optimal composition (60% formamide, 6×SSC, 0.15 M sucrose, 0.8% Ficoll type 400, 0.01% Triton X-100, 10% dextran sulfate), 1 L of target sequence or water (for negative control), 1 L of bead suspension containing 1 gram of beads, and 0.25 L of liposomes encapsulating sulforhodamine B dye molecules were incubated for 15 minutes at room temperature in a shaker. Following incubation, the mixture was loaded into the microfluidic channel (see FIG. 12) through inlet 1 at a flow rate of 14 L/minute. The liposome-target sequence-bead complexes formed were captured by the magnet in the detection zone. After all of the beads with the specific complexes were collected on the magnet and unbound liposomes were washed away with running buffer, the fluorescence image of intact liposomes was detected as described below. For the quantification of lysed liposomes, a 30 mM OG solution was continuously injected through inlet 2 in order to lyse the liposomes completely and release the sulforhodamine B dye molecules into the microchannel.

The fluorescence of intact and lysed liposomes was visualized using a Leica DMLB microscope (Leica Microsystems, Wetzlar, Germany) with a setup as follows: a 10×/0.25 NA long working distance objective, the appropriate filter set (540/25 nm band-pass exciter; 620/60 nm band-pass emitter), and 100-W mercury illumination source. The images of beads in the detection zone were obtained with a digital CoolSnap CCD camera (Photometrics, Tucson, Ariz.) coupled to image acquisition software (Roper Scientific Inc., Tucson, Ariz.). The fluorescence was quantified using Image ProExpress software (Media Cypernetics, Silver Spring, Md.).

### Example 3

### Immobilization of Dendrimers in Detection Region of Device

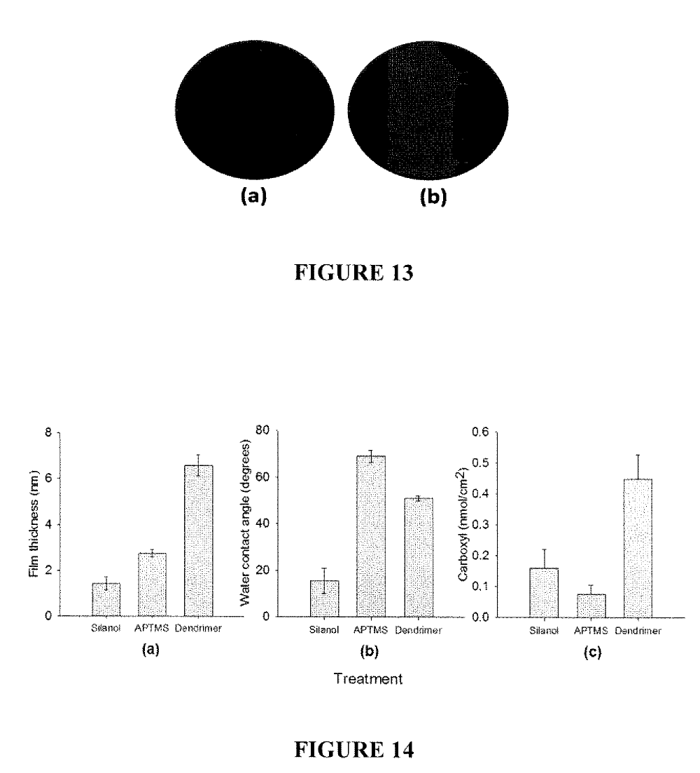

Generation 3.5 dendrimers were immobilized in the detection region of the microfluidic biosensor including a PMMA substrate. The functionalized ends of the dendrimer were then conjugated to DNA capture probes. These probes were used to immobilize target DNA while dye encapsulating liposomes served as a reporter probe by binding to another sequence on the target. Fluorescent images of the capture zone prior to binding, following binding, and then following liposome lysis show a successful detection of the target analyte (FIG. 13).

Dendrimer immobilization and capture was also demonstrated on a silicon substrate. The substrate was initially functionalized with (3-aminopropyl) trimethoxysilane (APTMS) followed by carboxylic-acid-terminated dendrimers. Analysis of the surface layers was conducted with a Imaging Ellipsometer (Nanofilm, Göttingen, Germany) for film thickness, VCA Optima XE (AST Products, Bellerica, Mass.) for water contact angle, and a toluidine blue O dye test for carboxylic acid content (FIG. 14).

### Example 4

### Surface Treatment of poly(methylmethacrylate) (PMMA)

PMMA is commonly used for micro fluidic devices due their low cost, optical clarity and ideal thermal and mechanical properties. The use of PMMA in these devices has created interest in the surface modification of PMMA. Oxidation of PMMA results in the formation of carboxylic acids by cleavage of a methylester bond. Conjugation to the surface carboxylic acids can then be accomplished using water soluble carbodiimide chemistry. Therefore, the initial carboxylic acid formation and density become very important. Several methods have been investigated for initial carboxylic acid formation. These methods include UV irradiation, O₃ exposure, and a combination of the two. Other wet methods such as H₂SO₄ hydrolysis have also been shown to provide surface carboxylic acids.

The following experiments investigated the use of UV, O₃, UV/O₃, and corona discharge as carboxylic acid formation methods. Corona discharge was investigated due to its ability to form O₃. PMMA chips, approximately 6 mm in diameter were used in these experiments. All chips were sonicated in 50% 2-propanol for 10 minutes, rinsed with DI water, and dried with N₂ prior to use.

The UV, O₃, and UV/O₃ treatments were conducted in a UV/ozone stripper (SAMCO International, Inc., Sunnyvale, Calif.) at ambient temperatures and a distance of 10 mm from the bulb (10 mW/cm² at 254 nm). The O₂ flow rate during all processes was 5 L/minute. The corona discharge experiment used a high frequency generator tesla coil (Electro-Technic Products, Inc., Chicago, Ill.) with the tip suspended 10 mm above the PMMA. All treatments had a duration of ten minutes.

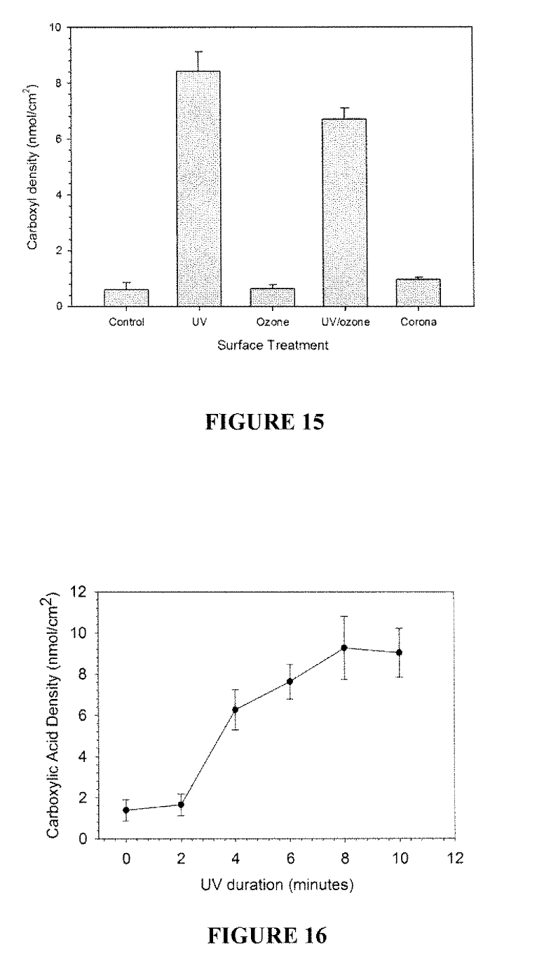

The results of the experiments indicated that the UV generated a much higher carboxylic acid density than the other surface treatments (FIG. 15). The UV/O₃ treatment resulted in less carboxylic acid formation than UV alone. This is most likely due to the partial blocking of UV light by O₃ molecules.

The effect of UV duration on the carboxylic acid formation was also investigated. The experiment was conducted similar to that previously described for UV treatment although the duration was 0, 2, 4, 6, 8, and 10 minutes. Again, the TBO dye assay was used for quantification of the carboxylic acids.

The results indicate a peak in carboxylic acid formation at approximately eight minutes (FIG. 16). After eight minutes of UV treatment, the carboxylic acid formation declines slightly most likely due to main chain scission resulting in etching of the PMMA surface. At this point the PMMA surface is etching at a rate faster than carboxylic acid formation. This should result in the formation of hydrophilic, low molecular weight polymers on the PMMA surface.

The formation of carboxylic acids on the surface, in addition to the removal of a methyl group, results in the surface becoming more hydrophilic. The hydrophilicity of the surface can be quantified by water contact angle.

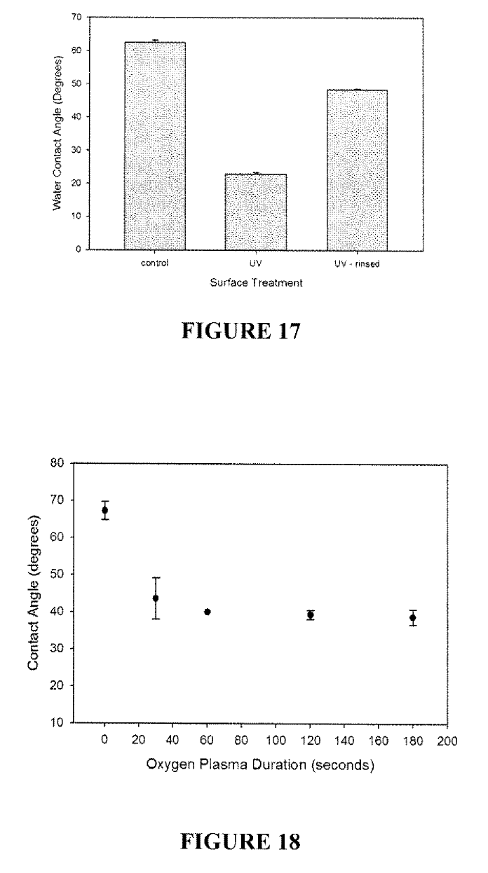

Cleaned PMMA discs were UV treated for ten minutes and then compared to native PMMA. The contact angle was measured using a VCA Optima XE goniometer (AST Products Inc., Boston, Mass.). The result was a significant drop in water contact angle from 62.5° to 22.9°. When the PMMA was rinsed for ten minutes with DI water following the UV treatment, the contact angle was measured at 48.4° (FIG. 17). This is consistent with previous published reports which conclude that the main chain scission and side chain modification results in lower molecular weight soluble polymer formation of the surface of the UV-treated PMMA.

In addition to UV treatment, the effect of O₂ plasma on the water contact angle of PMMA was also investigated. For this experiment, the PMMA was treated in a PlasmaTherm RIE SLR-720 (St. Petersburg, Fla.) using 150 sccm O₂ and 200 W.

The cleaned samples were placed in the reaction chamber for durations of 0, 30, 60, 120 and 180 seconds. The hydrophilicity of the samples was then immediately measured. There appeared to be an immediate drop in contact angle after only 30 seconds of O₂ plasma treatment followed by a steady leveling off (FIG. 18). Unlike the UV-treated samples, the O₂ plasma-treated samples were not able to be thermally bonded below the glass transition temperature of the bulk PMMA suggesting fewer low molecular weight polymers on the surface due to etching.

The use of UV-treatment toward the carboxylation formation on the surface appears to have an optimal functionalization time of approximately eight minutes. At this point, the carboxylic acid density is approximately 8-9 nmol/cm² and the main chain scission is adequate for reduced T_{g}. The reduced T_{g} of the surface allows the PMMA to be bonded below the T_{g} of the bulk PMMA. Therefore, UV treatment of PMMA channels for eight minute durations at 10 mW/cm² was used for future bonding of PMMA microchannels. Once rinsed, the bonded devices should contain 8-9 nmol/cm² of carboxylic acids on the channel surfaces. The carboxylic acids can be used to conjugate amine-modified poly(ethyleneglycol), DNA, or other biomolecules. The carboxylic acids also provide a negatively charged surface, which could aid in electro osmotic flow, or reduced adsorption of negatively charged markers such as liposomes. The use of UV is also considered a clean functionalization procedure. Other polymer functionalization methods using strong oxidizers such as H₂SO₄, chromic acid, or nitric acid result in hazardous waste.

Highly sensitive detection of nucleic acid molecules using interdigitated ultramicroelectrode arrays (IDUA) fabricated on Pyrex® 7740 as a substrate and overlayed polydimethylsiloxane (PDMS) channels has been previously demonstrated. Here, a titanium adhesion layer was used between the gold and substrate surface. In general, gold has poor adhesion properties to most surfaces including PMMA. Thus, an intermediate adhesion layer of another metal such as titanium or chromium is commonly used between the substrate and the gold layer. However, for an electrochemical detection system, a bimetallic system results in a galvanic cell with the less noble of the two metals being solubilized. Since it cannot be guaranteed that the adhesion layer is not coming in contact with the solution, it can result in limited lifetime of the electrode. Alternatively, mercaptopropyltrimethoxysilane (MPTMS) has been used as an effective alternative to a metallic adhesion layer by providing the substrate surface with a thiol monolayer. The gold electrodes are then adhered using gold-thiol interactions. Although limitations were found for the usable applied potential, the electrodes were more stable and had a longer lifetime compared to metallic adhesion systems.

In this study, an improved method for adhesion was found. In particular, cystamine was conjugated to the UV-modified PMMA surface using water soluble carbodiimide chemistries, resulting in a thiolated surface. A liposomal detection system was employed to aid in signal amplification. The liposome was tagged with a DNA probe complimentary to the target RNA. Superparamagnetic beads tagged with a target complimentary capture probe were used to immobilize the target and liposome complex over the IDUA.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A microchannel device for detection or quantification of an analyte in a sample, said device comprising:
a substrate defining one or more microchannels and having a reaction region in a first portion of the one or more microchannels, wherein the reaction region comprises a first binding element selected to bind with a first portion of the analyte; and
a detection region in fluid communication with the reaction region, said detection region comprising a second binding element selected to immobilize the analyte within the detection region.

2. The microchannel device according to claim 1 further comprising a detection device proximate the detection region for determining the presence or amount of analyte in the sample.

3. The microchannel device according to claim 2, wherein the detection device is an electrochemical detection device comprising one or more electrodes positioned to detect a change in electrical current within the detection region.

4. The microchannel device according to claim 3, wherein the one or more electrodes are gold.

5. The microchannel device according to claim 4, wherein the one or more electrodes are attached to the device by one or more adhesive layers comprising a heterobifunctional molecule comprising an amino group and a thiol group.

6. The microchannel device according to claim 5, wherein the heterobifunctional molecule is selected from the group consisting of cystamine, cystine, and 3-(2-pyridyldithio) propiony hydaride (PDPH).

7. The microchannel device according to claim 2, wherein the detection device is an optical detection device.

8. The microchannel device according to claim 1, wherein the substrate is selected from the group consisting of polymers, glass, silicon, and ceramics.

9. The microchannel device according to claim 8, wherein the substrate is poly(methyl methacrylate).

10. The microchannel device according to claim 1, wherein the one or more microchannels comprise a micro fluidic mixer.

11. The microchannel device according to claim 1, wherein one or both of the first and second binding elements are immobilized to a surface of the reaction region and detection region.

12. The microchannel device according to claim 1, wherein one or both of the first and second binding elements are coupled to one or more magnetic beads.

13. The microchannel device according to claim 12 further comprising:
a magnet positioned proximate the reaction or detection region under conditions effective to retain the one or more magnetic beads within the reaction or detection region.

14. The microchannel device according to claim 1, wherein the detection region comprises one or more immobilized dendrimers, wherein one or more of the dendrimers comprise one or more second binding element.

15. The microchannel device according to claim 1, wherein the first and second binding elements are selected from the group consisting of antibodies, antigens, nucleic acid molecules, aptamers, cell receptors, biotin, and streptavidin.

16. The microchannel device according to claim 15, wherein the first and second binding elements are nucleic acid molecules designed to bind specifically with distinct portions of the analyte.

17. A method of detecting or quantifying an analyte in a test sample, said method comprising:
providing a device comprising:
a substrate defining one or more microchannels and having a reaction region in a first portion of the one or more microchannels; and
a detection region in fluid communication with the reaction region;
introducing a test sample potentially comprising a target analyte into the reaction region, under conditions effective to permit binding between a first binding element present within the reaction region and a first portion of the analyte;
providing a second binding element selected to immobilize the analyte within the detection region, wherein the second binding element is capable of binding with a second portion of the analyte or a portion of the first binding element;
contacting the test sample with the detection region, under conditions effective to immobilize the analyte within the detection region;
providing one or more reporter complexes under conditions effective to permit binding between the reporter complexes and a third portion of the analyte or a portion of the first binding element or a portion of the second binding element;
detecting any reporter complexes bound to the analyte or first binding element or second binding element in the detection region; and
correlating the presence or quantity of bound reporter complexes to the presence or quantity of analyte in the test sample.

18. The method according to claim 17, wherein the analyte is selected from the group consisting of antigens, haptens, cells, and target nucleic acid molecules.

19. The method according to claim 17, wherein the substrate is selected from the group consisting of polymers, glass, silicon, and ceramics.

20. The method according to claim 17, wherein the one or more microchannels comprise a micro fluidic mixer.

21. The method according to claim 17 further comprising:
immobilizing the first binding element within the reaction region.

22. The method according to claim 21, wherein the first binding element is immobilized to a surface of the reaction region.

23. The method according to claim 17, wherein the first binding element is coupled to one or more magnetic beads.

24. The method according to claim 23 further comprising:
contacting the device with a magnetic force before or during any of said introducing, providing, and contacting steps under conditions effective to retain the one or more magnetic beads within the reaction region.

25. The method according to claim 17 further comprising:
immobilizing the second binding element within the detection region.

26. The method according to claim 25, wherein the second binding element is immobilized to a surface of the detection region.

27. The method according to claim 17, wherein the second binding element is coupled to one or more magnetic beads.

28. The method according to claim 27 further comprising:
contacting the device with a magnetic force before or during any of said introducing, providing, contacting, and detecting steps under conditions effective to retain the magnetic beads within the detection region.

29. The method according to claim 17, wherein the detection region comprises one or more immobilized dendrimers, wherein one or more of the dendrimers comprise one or more second binding element.

30. The method according to claim 17, wherein one or both of the first and second binding elements are provided in solution with the test sample.

31. The method according to claim 17, wherein the first and second binding elements are selected from the group consisting of antibodies, antigens, nucleic acid molecules, aptamers, cell receptors, biotin, and streptavidin.

32. The method according to claim 17, wherein the analyte is a nucleic acid, said method further comprising:
amplifying the analyte in the reaction region prior to said contacting.

33. The method according to claim 17, wherein the one or more reporter complexes are introduced into the reaction region prior to said contacting.

34. The method according to claim 17, wherein the one or more reporter complexes are introduced into the detection region.

35. The method according to claim 17, wherein detecting comprises positioning an electrochemical detection device proximate the detection region under conditions effective to detect any reporter complexes bound to the analyte or second binding element.

36. The method according to claim 35, wherein the electrochemical detection device comprises one or more electrodes positioned to detect a change in electrical current within the detection region.

37. The method according to claim 36, wherein the one or more electrodes are gold.

38. The method according to claim 37, wherein the one or more electrodes are attached to the device by one or more adhesive layers comprising a heterobifunctional molecule comprising an amino group and a thiol group.

39. The method according to claim 38, wherein the heterobifunctional molecule is selected from the group consisting of cystamine, cystine, and 3-(2-pyridyldithio) propiony hydaride (PDPH).

40. The method according to claim 17, wherein detecting comprises positioning an optical detection device proximate the detection region under conditions effective to detect any reporter complexes bound to the analyte or second binding element.

41. The method according to claim 17, wherein the one or more reporter complexes comprise a liposome containing a detectable label.

42. A method of detecting or quantifying an analyte in a test sample, said method comprising:
providing a device comprising:
a substrate defining one or more microchannels and having a reaction region in a first portion of the one or more microchannels; and
a detection region in fluid communication with the reaction region;
introducing a test sample potentially comprising a target analyte into the reaction region;
providing one or more reporter complexes, wherein the reporter complexes comprise a first binding element and a marker, under conditions effective to permit binding between the first binding element and a first portion of the analyte;
providing a second binding element selected to immobilize the analyte within the detection region, under conditions effective to permit binding between the second binding element and a second portion of the analyte or a portion of the one or more reporter complexes;
contacting the test sample and the one or more reporter complexes with the detection region, under conditions effective to immobilize the analyte within the detection region;
detecting any reporter complexes bound to the analyte in the detection region; and
correlating the presence or quantity of bound reporter complexes to the presence or quantity of analyte in the test sample.

43. The method according to claim 42, wherein the analyte is selected from the group consisting of antigens, haptens, cells, and target nucleic acid molecules.

44. The method according to claim 42, wherein the substrate is selected from the group consisting of polymers, glass, silicon, and ceramics.

45. The method according to claim 42, wherein the one or more microchannels comprise a micro fluidic mixer.

46. The method according to claim 42, wherein at least one of the one or more reporter complexes and the second binding element are provided in solution with the test sample in the reaction region.

47. The method according to claim 42, wherein at least one of the one or more reporter complexes and the second binding element are introduced in the detection region.

48. The method according to claim 42, further comprising:
immobilizing the second binding element within the detection region.

49. The method according to claim 48, wherein the second binding element is immobilized to a surface of the detection region.

50. The method according to claim 42, wherein the second binding element is coupled to one or more magnetic beads.

51. The method according to claim 50 further comprising:
contacting the device with a magnetic force before or during any of said introducing, providing, contacting, and detecting steps under conditions effective to retain the magnetic beads within the detection region.

52. The method according to claim 42, wherein the detection region comprises one or more immobilized dendrimers, wherein one or more of the dendrimers comprise one or more second binding element.

53. The method according to claim 42, wherein the first and second binding elements are selected from the group consisting of antibodies, antigens, nucleic acid molecules, aptamers, cell receptors, biotin, and streptavidin.

54. The method according to claim 42, wherein detecting comprises positioning an electrochemical detection device proximate the detection region under conditions effective to detect any reporter complexes bound to the analyte.

55. The method according to claim 54, wherein the electrochemical detection device comprises one or more electrodes positioned to detect a change in electrical current within the detection region.

56. The method according to claim 55, wherein the one or more electrodes are gold.

57. The method according to claim 56, wherein the one or more electrodes are attached to the device by one or more adhesive layers comprising a heterobifunctional molecule comprising an amino group and a thiol group.

58. The method according to claim 57, wherein the heterobifunctional molecule is selected from the group consisting of cystamine, cystine, and 3-(2-pyridyldithio) propiony hydaride (PDPH).

59. The method according to claim 42, wherein detecting comprises positioning an optical detection device proximate the detection region under conditions effective to detect any reporter complexes bound to the analyte.

60. The method according to claim 42, wherein the one or more reporter complexes comprise a liposome containing a detectable label.

61. A method for coating a polymer with a gold layer comprising:
providing a polymer having at least a portion of a surface having a plurality of carboxylic acids;
conjugating a heterobifunctional molecule containing an amino group and a thiol group to the surface under conditions effective to produce a thiolated surface; and
adhering a gold layer to the thiolated surface.

62. The method according to claim 61, wherein the polymer is selected from the group consisting of polyethylene, polypropylene, poly(4-methylbutene), polystyrene, polycarbonate, poly(methyl methacrylate), poly(ethylene terephthalate), nylon, poly(vinyl chloride), and poly(vinyl butyrate).

63. The method according to claim 62, wherein the polymer is poly(methyl methacrylate).

64. The method according to claim 61, wherein the heterobifunctional molecule is selected from the group consisting of cystamine, cystine, and 3-(2-pyridyldithio) propiony hydaride (PDPH).

65. The method according to claim 61, wherein providing comprises treating at least a portion of the surface of the polymer under conditions effective to form a plurality of carboxylic acids on the surface.

66. The method according to claim 65, wherein treating comprises UV irradiation, O₃ exposure, UV/O₃ combination, H₂SO₄ hydrolysis, or corona discharge.

67. The method according to claim 65, wherein treating results in a carboxylic acid density of from about 0.1 nmol/cm² to about 100 nmol/cm² on the surface.
